SEQUENCE LISTING (34 sequences)

SEQ ID NO: 1 - protein, 15 aa, Artificial sequence
  Pembrolizumab-Light chain CDR1
RASKGVSTSGYSYLH

SEQ ID NO: 2 - protein, 7 aa, Artificial Sequence
  Pembrolizumab-Light chain CDR2
LASYLES

SEQ ID NO: 3 - protein, 9 aa, Artificial Sequence
  Pembrolizumab-Light chain CDR3
QHSRDLPLT

SEQ ID NO: 4 - protein, 111 aa, Artificial Sequence [sequence omitted]

SEQ ID NO: 5 - protein, 218 aa, Artificial Sequence
  Pembrolizumab-Light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  Pembrolizumab-Heavy chain CDR1
NYYMY

SEQ ID NO: 7 - protein, 17 aa, Artificial sequence
  Pembrolizumab-Heavy chain CDR2
GINPSNGGTNFNEKFKN

SEQ ID NO: 8 - protein, 11 aa, Artificial sequence
  Pembrolizumab-Heavy chain CDR3
RDYRFDMGFDY

SEQ ID NO: 9 - protein, 120 aa, Artificial sequence [sequence omitted]

SEQ ID NO: 10 - protein, 447 aa, Artificial sequence
  Pembrolizumab-Heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 11 aa, Homo sapiens
RASQSVSSYLA

SEQ ID NO: 12 - protein, 7 aa, Homo sapiens
DASNRAT

SEQ ID NO: 13 - protein, 9 aa, Homo sapiens
QQSSNWPRT

SEQ ID NO: 14 - protein, 107 aa, Homo sapiens [sequence omitted]

SEQ ID NO: 15 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, Homo sapiens
NSGMH

SEQ ID NO: 17 - protein, 17 aa, Homo sapiens
VIWYDGSKRYYADSVKG

SEQ ID NO: 18 - protein, 4 aa, Homo sapiens
NDDY

SEQ ID NO: 19 - protein, 113 aa, Homo sapiens [sequence omitted]

SEQ ID NO: 20 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 15 aa, Artificial sequence
  hPD-1.08A Light Chain CDR1
RASKSVSTSGFSYLH

SEQ ID NO: 22 - protein, 7 aa, Artificial sequence
  hPD-1.08A Light Chain CDR2
LASNLES

SEQ ID NO: 23 - protein, 9 aa, Artificial sequence
  hPD-1.08A Light Chain CDR3
QHSWELPLT

SEQ ID NO: 24 - protein, 5 aa, Artificial sequence
  hPD-1.08A Heavy Chain CDR1
SYYLY

SEQ ID NO: 25 - protein, 17 aa, Artificial sequence
  hPD-1.08A Heavy Chain CDR2
GVNPSNGGTNFSEKFKS

SEQ ID NO: 26 - protein, 11 aa, Artificial sequence
  hPD-1.08A Heavy Chain CDR3
RDSNYDGGFDY

SEQ ID NO: 27 - protein, 120 aa, Artificial sequence [sequence omitted]

SEQ ID NO: 28 - protein, 111 aa, Artificial sequence [sequence omitted]

SEQ ID NO: 29 - protein, 111 aa, Artificial sequence
  K09A light chain variable region
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 30 - protein, 111 aa, Artificial sequence
  K09A light chain variable region
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 31 - protein, 447 aa, Artificial sequence; identical to SEQ ID NO: 10
  mature 409 heavy chain

SEQ ID NO: 32 - protein, 218 aa, Artificial sequence; identical to SEQ ID NO: 5
  mature K09A light chain

SEQ ID NO: 33 - protein, 218 aa, Artificial sequence
  mature K09A light chain
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 218 aa, Artificial sequence
  mature K09A light chain
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0380694 A1
(43) Pub. Date: Dec. 9, 2021

(54) STABLE FORMULATIONS OF PROGRAMMED DEATH RECEPTOR 1 (PD-1) ANTIBODIES AND METHODS OF USE THEREOF

(71) Applicants: William P. FORREST, JR., Kenilworth, NJ (US); Chakravarthy Nachu NARASIMHAN, Kenilworth, NJ (US); Yogita KRISHNAMACHARI, Kenilworth, NJ (US); Merck Sharp & Dohme Corp., Rahway, NJ (US)

(72) Inventors: William P. Forrest, Jr., Middletown, NJ (US); Chakravarthy Nachu Narasimhan, Scotch Plains, NJ (US); Yogita Krishnamachari, Scotch Plains, NJ (US)

(73) Assignee: Merck Sharp & Dohme Corp., Rahway, NJ (US)

(21) Appl. No.: 17/290,816

(22) PCT Filed: Nov. 6, 2019
(86) PCT No.: PCT/US2019/059957

§ 371 Date: May 3, 2021

Related U.S. Application Data
(60) Provisional application No. 62/756,948, filed on Nov. 7, 2018.

(51) Int. Cl. C07K 16/28; A61K 47/10; A61K 47/18; A61K 47/26; A61K 47/40; A61P 35/00

(52) U.S. Cl. CPC C07K 16/2818; A61K 47/10; A61K 47/183; A61P 35/00; A61K 47/26; A61K 47/40; A61K 47/186

(57) ABSTRACT

The invention relates to stable formulations of antibodies against human programmed death receptor PD-1, or antigen binding fragments thereof. In some embodiments the formulations of the invention comprise between 5-250 mg/mL anti-PD-1 antibody, or antigen binding fragment thereof, a buffer, a stabilizer, a surfactant, and an antioxidant in the amounts specified herein. In particular embodiments, the anti-PD-1 antibody is pembrolizumab. The invention further provides methods for treating various cancers with stable formulations of the invention. In some embodiments of the methods of the invention, the formulations are administered to a subject by intravenous or subcutaneous administration.

### CROSS-REFERENCE TO RELATED APPLICATIONS

This application claims the benefit of U.S. Provisional Application No. 62/756,948, filed Nov. 7, 2018, the content of which is hereby incorporated by reference in its entirety.

### FIELD OF THE INVENTION

The invention relates to stable formulations comprising antibodies or antigen binding fragments thereof that bind to human programmed death receptor 1 (PD-1). Also provided are methods of treating various cancers and chronic infections with the formulations of the invention.

### REFERENCE TO SEQUENCE LISTING SUBMITTED ELECTRONICALLY

The sequence listing of the present application is submitted electronically via EFS-Web as an ASCII formatted sequence listing with a file name “24657WOPCT-SEQTXT-01NOV2019.TXT”, creation date of Nov. 1, 2019, and a size of 33.1 Kb. This sequence listing submitted via EFS-Web is part of the specification and is herein incorporated by reference in its entirety.

### BACKGROUND OF THE INVENTION

Immune checkpoint therapies targeting the programmed death receptor-1 (PD-1) axis have resulted in groundbreaking improvements in clinical response in multiple human cancers (Brahmer et al., N Engl J Med 2012, 366: 2455-65; Garon et al. N Engl J Med 2015, 372: 2018-28; Hamid et al., N Engl J Med 2013, 369: 134-44; Robert et al., Lancet 2014, 384: 1109-17; Robert et al N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; Wolchok et al., N Engl J Med 2013, 369: 122-33). The interaction of the PD-1 receptor on T-cells with its ligands, PD-L1 and PD-L2, on tumor and immune infiltrating cells regulates T-cell mediated immune responses and may play a role in immune escape by human tumors (Pardoll D M. Nat Rev Cancer 2012, 12: 252-64). Binding of PD-1 to either of its ligands results in delivery of an inhibitory stimulus to the T cell. Immune therapies targeting the PD-1 axis include monoclonal antibodies directed to the PD-1 receptor (KEYTRUDA™ (pembrolizumab), Merck and Co., Inc., Kenilworth, N.J. and OPDIVO™ (nivolumab), Bristol-Myers Squibb, Princeton, N.J.) and also those that bind to the PD-L1 ligand (MPDL3280A; TECENTRIQ™ (atezolizumab), Genentech, San Francisco, Calif.). Both therapeutic approaches have demonstrated anti-tumor effects in numerous cancer types.

Antibodies for use in human subjects must be stored prior to use and transported to the point of administration. Reproducibly attaining a desired level of antibody drug in a subject requires that the drug be stored in a formulation that maintains the bioactivity of the drug. The need exists for stable formulations of anti-human PD-1 antibodies for pharmaceutical use, e.g., for treating various cancers and infectious diseases. Preferably, such formulations will exhibit a long shelf-life, be stable when stored and transported, and will be amenable to administration at high concentrations, e.g. for use in subcutaneous administration, as well as low concentrations, e.g. for intravenous administration.

### SUMMARY OF THE INVENTION

The invention provides an anti-human PD-1 antibody formulation, comprising: an anti-human PD-1 antibody formulation, comprising: a) about 5 mg/mL to about 250 mg/mL of an anti-human PD-1 antibody, or antigen binding fragment thereof; b) about 5 mM to about 20 mM buffer; c) about 1.5 to about 8.0% weight/volume (w/v) stabilizer selected from the group consisting of: a non-reducing sugar, (2-hydroxypropyl)β-cyclodextrin, mannitol, sorbitol, L-arginine, a pharmaceutically acceptable salt of L-arginine, L-proline, a pharmaceutically acceptable salt of L-proline, L-histidine, a pharmaceutically acceptable salt of L-histidine, glycine, or a pharmaceutically acceptable salt of glycine, d) a surfactant selected from about 0.005% w/v to about 0.60% w/v non-ionic surfactant and about 0.23% w/v to about 1.15% w/v ionic surfactant; and e) about 1 mM to about 30 mM anti-oxidant.

In some embodiments, the stabilizer is a non-reducing sugar which is sucrose or trehalose.

In specific embodiments, the stabilizer is selected from the group consisting of: (i) about 6% to about 8% weight/volume (w/v) sucrose, trehalose or (2-hydroxypropyl)-O-cyclodextrin; (ii) about 3% to about 5% w/v mannitol, sorbitol, L-arginine, a pharmaceutically acceptable salt of L-arginine, L-proline, or a pharmaceutically acceptable salt of L-proline; (iii) about 1.8% to about 2.2% w/v glycine, or a pharmaceutically acceptable salt thereof; (iv) about 1.5% to 1.9% w/v L-proline, or a pharmaceutically acceptable sale of L-proline; (v) about 1.9%-3.3% w/v L-arginine, or a pharmaceutically acceptable salt of L-arginine; and (vi) about 2% to about 3% L-histidine, or a pharmaceutically acceptable salt of L-histidine; and the antioxidant is about 1 mM to about 20 mM L-methionine or a pharmaceutically acceptable salt thereof.

In specific embodiments, the stabilizer is about 1.5% to 1.9% w/v L-proline, or a pharmaceutically acceptable sale of L-proline.

In specific embodiments, the stabilizer is about 1.7% w/v L-proline, or a pharmaceutically acceptable sale of L-proline.

Some embodiments comprise greater than 200 mg/mL of the anti-human PD-1 antibody, or antigen binding fragment thereof.

In some embodiments, the surfactant is: (a) about 0.23% w/v to about 1.15% w/v sodium dodecyl sulfate; (b) about 0.005% w/v to about 0.60% w/v non-ionic surfactant, which is selected from the group consisting of: polysorbate 20, a poloxamer, vitamin E D-α-tocopherol polyethylene glycol succinate (TPGS), polyethylene glycol tert-octylphenyl ether, and n-octyl β-D-maltoside (OM), or (c) about 0.005% w/v to about 0.20% w/v dimethyl-dodecylamine oxide (DDAO).

In some embodiments, the surfactant is: (a) about 0.23% w/v to about 1.15% w/v sodium dodecyl sulfate; (b) about 0.005% w/v to about 0.60% w/v non-ionic surfactant, which is selected from the group consisting of: poloxamer 338 (P338), poloxamer 407 (P407), vitamin E D-α-tocopherol polyethylene glycol succinate (TPGS), n-dodecyl β-D-maltoside (DDM) and n-octyl β-D-maltoside (OM), or (c) about 0.005% w/v to about 0.20% w/v dimethyl-dodecylamine oxide (DDAO).

In specific embodiments, the surfactant is about 0.01% to about 0.03% w/v poloxamer 338 (P338).

In specific embodiments, the surfactant is about 0.01% to about 0.03% w/v poloxamer 407 (P407).

In specific embodiments, the surfactant is about 0.01% to about 0.03% w/v vitamin E D-α-tocopherol polyethylene glycol succinate (TPGS).

In specific embodiments, the surfactant is about 0.01% to about 0.03% w/v n-dodecyl β-D-maltoside (DDM).

In specific embodiments, the surfactant is about 0.4% to about 0.6% w/v n-octyl β-D-maltoside (OM).

In specific embodiments, the surfactant is about 0.01% to about 0.03% w/v dimethyl-dodecylamine oxide (DDAO).

In specific embodiments, the buffer is a histidine buffer or an acetate buffer.

In some embodiments, the anti-oxidant is L-methionine, which is present at a concentration of about 1 mM to about 20 mM.

In further embodiments, the invention provides an anti-human PD-1 antibody formulation which comprises: (a) about 200 mg/mL to about 250 mg/mL of an anti-human PD-1 antibody, or antigen binding fragment thereof; (b) about 5 mM to about 20 mM histidine buffer; (c) a stabilizer selected from the group consisting of: (i) about 6% to about 8% weight/volume (w/v) sucrose, trehalose or (2-hydroxypropyl)-β-cyclodextrin; (ii) about 3% to about 5% w/v mannitol, sorbitol, L-arginine, a pharmaceutically acceptable salt of L-arginine, L-proline, or a pharmaceutically acceptable salt of L-proline; (iii) about 1.8 to about 2.2% w/v glycine, or a pharmaceutically acceptable salt thereof; (iv) about 1.5% to 1.9% w/v L-proline, or a pharmaceutically acceptable sale of L-proline; (v) about 1.9%-3.3% w/v L-arginine, or a pharmaceutically acceptable salt of L-arginine; and (vi) about 2% to about 3% L-histidine, or a pharmaceutically acceptable salt of L-histidine; (d) about 0.01% w/v to about 0.10% w/v polysorbate 80; and (e) about 1 mM to about 20 mM L-methionine, or a pharmaceutically acceptable salt thereof.

In particular embodiments, the formulation further comprises from about 1% to about 3% w/v of a viscosity reducing agent such as, but not limited to, L-arginine, or a pharmaceutically acceptable salt thereof, L-lysine, or a pharmaceutically acceptable thereof, L-histidine, or a pharmaceutically acceptable thereof, and L-glutamine, or a pharmaceutically acceptable thereof. In some embodiments, the formulation comprises two or more viscosity reducing agents.

In specific embodiments of the formulations of the invention, the surfactant is: a surfactant selected from: (i) about 0.005% w/v to about 0.60% w/v non-ionic surfactant; (ii) about 0.23% w/v to about 1% w/v ionic surfactant; or (iii) about 0.005% w/v to about 0.20% w/v dimethyl-dodecylamine oxide (DDAO); and In embodiments of the invention, the buffer provides a pH of between 5.0 and 6.0.

In specific embodiments, the stabilizer of the anti-human PD-1 antibody formulation is selected from the group consisting of: (i) about 6% to about 8% w/v sucrose, trehalose or (2-hydroxypropyl)-β-cyclodextrin; (ii) about 3% to about 5% mannitol, sorbitol, or L-proline, or a pharmaceutically acceptable salt of L-proline; and (iii) about 1.8 to about 2.2% w/v glycine, or a pharmaceutically acceptable salt thereof.

In certain embodiments, the anti-human PD-1 antibody formulation further comprises a metal chelator. In specific embodiments, the metal chelator is DTPA. In certain embodiments the DTPA is present at a concentration of about 10 μM to about 30 μM.

The invention also provides a liquid anti-human PD-1 antibody formulation that is reconstituted from a lyophilized formulation wherein the reconstituted solution comprises: a) about 125 mg/mL to about 175 mg/mL of an anti-human PD-1 antibody, or antigen binding fragment thereof; b) about 8 mM to about 12 mM histidine buffer; c) a stabilizer selected from the group consisting of: (i) about 3% to about 8% w/v sucrose; (ii) about 2% to about 5% w/v L-arginine, or a pharmaceutically acceptable salt thereof; (iii) about 3% to about 5% mannitol and about 1% to about 2% sucrose; and (iv) a combination of i) and ii); and d) about 0.01% to about 0.04% polysorbate 80.

In specific embodiments of the invention the anti-PD-1 antibody is pembrolizumab, a pembrolizumab variant, or an antigen binding fragment of pembrolizumab.

Also provided herein are methods of treating cancer and methods of treating chronic infection in a human patient in need thereof comprising: administering an effective amount of the anti-human PD-1 antibody formulations of the invention to the patient.

### DETAILED DESCRIPTION OF THE INVENTION

The invention provides stable formulations comprising an anti-PD-1 antibody, or antigen binding fragment thereof that binds to human PD-1, which are useful for methods of treatment of cancer or an immune disorder or immune condition which comprise administration to a patient in need thereof for example, by intravenous or subcutaneous administration. In certain embodiments of the invention, the anti-PD-1 antibody is pembrolizumab or an antigen binding fragment of pembrolizumab. The formulations of the invention address the issues of high viscosity and increased aggregation associated with antibody formulations comprising a high concentration of anti-PD1 antibodies. The invention further provides formulations comprising pembrolizumab or an antigen binding fragment thereof with reduced methionine oxidation, including reduced oxidation of methionine-105, which is located in CDR3 of the heavy chains of pembrolizumab.

The formulations of the invention are useful for subcutaneous delivery to a patient in need thereof. In order to deliver maximum therapeutic benefits to patients, it is desirable that formulations for subcutaneous (SC) delivery comprise a high antibody concentration (75-250 mg/mL). A high concentration of API is often required for SC formulations due to the historical bioavailability of 50-60% for SC injections and the expected dose range of an antibody product. However, high concentration of antibody, or antigen binding fragment thereof, may contribute to other properties of the product which would be undesirable, e.g. low injectability due to increased viscosity and higher than physiological osmolality and increased aggregation. Therefore, it is preferred that an antibody product intended for SC administration balances the effects of concentration while maintaining a level of drug that will provide the highest therapeutic benefit. An ideal product comprises a high protein concentration, low viscosity, an osmolality similar to physiological conditions, and a low level of aggregation under typical storage conditions. Increased viscosity at high protein concentration may not only make it difficult to extract the product from its container with a syringe, but also to inject the necessary dose into a patient from the syringe (syringeability). Advantageously, embodiments of the invention provide formulations that comprise a high concentration of antibody, or antigen binding fragment thereof, and a viscosity level that is acceptable for subcutaneous delivery. Additionally, the formulations of the invention do not lead to high levels of aggregation, as shown in more detail throughout the Examples.

Previous forced degradation studies were conducted on pembrolizumab drug substance (DS) to investigate product degradation pathways and to isolate and characterize impurities. In these studies, pembrolizumab DS was exposed to various stress conditions, and analysis of stressed samples indicated that, under the stress conditions employed, pembrolizumab DS was sensitive to light, peroxide, and high pH. Major degradation pathways of pembrolizumab included oxidation of methionine 105 (Met105) in the heavy chain CDR upon peroxide stress and oxidation of Met105 and Fc methionine residues when exposed to light. Pembrolizumab maintained its bioactivity under most stress conditions for the degradation levels tested. However, reduction in affinity to PD-1 was observed for peroxide stressed samples by Surface Plasmon Resonance (SPR). An exposed methionine residue or a methionine residue in the CDR of an antibody has the potential of impacting the biological activity of the antibody through oxidation. It is shown herein that the formulations of the invention are able to reduce oxidation of Met105 within the pembrolizumab heavy chain CDR.

### I. Definitions and Abbreviations

As used throughout the specification and appended claims, the following abbreviations apply:
API active pharmaceutical ingredient
CDR complementarity determining region in the immunoglobulin variable regions
CE-SDS capillary electrophoresis-sodium dodecyl sulfate
CHO Chinese hamster ovary
CI confidence interval
cP centipoise
DDAO dimethyl-dodecylamine oxide
DDM n-dodecyl β-D-maltoside
DS drug substance
EC50 concentration resulting in 50% efficacy or binding
ELISA enzyme-linked immunosorbant assay
FFPE formalin-fixed, paraffin-embedded
FR framework region
HC heavy chain
HNSCC head and neck squamous cell carcinoma
HPBC 2-Hydroxypropyl)-β-cyclodextrin
HP-HIC high performance hydrophobic interaction chromatography
HP-IEX high performance ion-exchange chromatography
HP-SEC high performance size exclusion chromatography
IC50 concentration resulting in 50% inhibition
IgG immunoglobulin G
IHC immunohistochemistry or immunohistochemical
mAb monoclonal antibody
MES 2-(N-morpholino)ethanesulfonic acid
NCBI National Center for Biotechnology Information
NSCLC non-small cell lung cancer
OM n-octyl β-D-maltoside
P188 poloxamer 188
P388 poloxamer 388
P407 poloxamer 407
PCR polymerase chain reaction
PD-1 programmed death 1 (a.k.a. programmed cell death-1 and programmed death receptor 1)
PD-L1 programmed cell death 1 ligand 1
PD-L2 programmed cell death 1 ligand 2
PS80 or PS-80 polysorbate 80
PS20 or PS-20 polysorbate 20
SBEC (sulfobutylether)-β-cyclodextrin
SWFI sterile water for injection
TNBC triple negative breast cancer
TPGS D-α-tocopherol polyethylene glycol succinate
VH immunoglobulin heavy chain variable region
VK immunoglobulin kappa light chain variable region
VL immunoglobulin light chain variable region
VP-DSC Valerian-Plotnikov differential scanning calorimetry
v/v volume per volume
WFI water for injection
w/v weight per volume

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

“Treat” or “treating” a cancer as used herein means to administer a formulation of the invention to a subject having an immune condition or cancerous condition, or diagnosed with a cancer or pathogenic infection (e.g. viral, bacterial, fungal), to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, stimulating an immune response to a pathogen, toxin, and/or self-antigen, stimulating an immune response to a viral infection, decreasing the number of one or more tumor markers, halting or delaying the growth of a tumor or blood cancer or progression of disease associated with PD-1 binding to its ligands PD-L1 and/or PD-L2 (“PD-1-related disease”) such as cancer, stabilization of PD-1-related disease, inhibiting the growth or survival of tumor cells, eliminating or reducing the size of one or more cancerous lesions or tumors, decreasing the level of one or more tumor markers, ameliorating, abrogating the clinical manifestations of PD-1-related disease, reducing the severity or duration of the clinical symptoms of PD-1-related disease such as cancer, prolonging the survival of a patient relative to the expected survival in a similar untreated patient, inducing complete or partial remission of a cancerous condition or other PD-1 related disease.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by administration of a formulation of the invention is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the formulations, treatment methods, and uses of the invention may not be effective in achieving a positive therapeutic effect in every patient, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

The term “patient” (alternatively referred to as “subject” or “individual” herein) refers to a mammal (e.g., rat, mouse, dog, cat, rabbit) capable of being treated with the formulations of the invention, most preferably a human. In some embodiments, the patient is an adult patient. In other embodiments, the patient is a pediatric patient. Those “in need of treatment” include those patients that may benefit from treatment with the formulations of the invention, e.g. a patient suffering from cancer or an immune condition.

The term “antibody” refers to any form of antibody that exhibits the desired biological activity. Thus, it is used in the broadest sense and specifically covers, but is not limited to, monoclonal antibodies (including full length monoclonal antibodies), polyclonal antibodies, humanized, fully human antibodies, and chimeric antibodies.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) Mol. Biol. 196:901-917 or Chothia, et al., (1989) Nature 342:878-883.

An antibody or antigen-binding fragment that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the invention will bind to the target protein with an affinity that is at least two-fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

The term “pharmaceutically effective amount” or “effective amount” means an amount whereby sufficient therapeutic composition or formulation is introduced to a patient to treat a diseased or condition. One skilled in the art recognizes that this level may vary according the patient's characteristics such as age, weight, etc.

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” can mean a variation of ±0.1%, 0.5%, 1%, 2%, 3%, 4%, 5% or 10%.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

A “chemotherapeutic agent” is a chemical compound useful in the treatment of cancer. Anti-PD-1 antibodies can be used with any one or more suitable chemotherapeutic agent. Examples of such chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

“Chothia” means an antibody numbering system described in A1-Lazikani et al., JMB 273:927-948 (1997).

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

A “growth inhibitory agent” when used herein refers to a compound or composition which inhibits growth of a cell, especially cancer cell over expressing any of the genes identified herein, either in vitro or in vivo. Thus, the growth inhibitory agent is one which significantly reduces the percentage of cells over expressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine) taxanes, and topo II inhibitors such as doxorubicin, epirubicin, daunorubicin, and etoposide. Those agents that arrest G1 also spill over into S-phase arrest, for example, DNA alkylating agents such as dacarbazine, mechlorethamine, and cisplatin. Further information can be found in The Molecular Basis of Cancer, Mendelsohn and Israel, eds., Chapter 1, entitled “Cell cycle regulation, oncogens, and antineoplastic drugs” by Murakami et al. (WB Saunders: Philadelphia, 1995).

The terms “PD-1 binding fragment,” “antigen binding fragment thereof,” “binding fragment thereof” or “fragment thereof” encompass a fragment or a derivative of an antibody that still substantially retains its biological activity of binding to antigen (human PD-1) and inhibiting its activity (e.g., blocking the binding of PD-1 to PDL1 and PDL2). Therefore, the term “antibody fragment” or PD-1 binding fragment refers to a portion of a full length antibody, generally the antigen binding or variable region thereof. Examples of antibody fragments include Fab, Fab′, F(ab′)2, and Fv fragments. Typically, a binding fragment or derivative retains at least 10% of its PD-1 inhibitory activity. In some embodiments, a binding fragment or derivative retains at least 25%, 50%, 60%, 70%, 80%, 90%, 95%, 99% or 100% (or more) of its PD-1 inhibitory activity, although any binding fragment with sufficient affinity to exert the desired biological effect will be useful. In some embodiments, an antigen binding fragment binds to its antigen with an affinity that is at least two fold greater, preferably at least ten times greater, more preferably at least 20-times greater, and most preferably at least 100-times greater than the affinity with unrelated antigens. In one embodiment the antibody has an affinity that is greater than about 10⁹ liters/mol, as determined, e.g., by Scatchard analysis. Munsen et al. (1980) Analyt. Biochem. 107:220-239. It is also intended that a PD-1 binding fragment can include variants having conservative amino acid substitutions that do not substantially alter its biologic activity.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The humanized forms of rodent antibodies will generally comprise the same CDR sequences of the parental rodent antibodies, although certain amino acid substitutions may be included to increase affinity, increase stability of the humanized antibody, or for other reasons.

The antibodies of the invention also include antibodies with modified (or blocked) Fc regions to provide altered effector functions. See, e.g., U.S. Pat. No. 5,624,821; WO2003/086310; WO2005/120571; WO2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta (2005) J. Allergy Clin. Immunol. 116:731 at 734-35.

“Fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody which comprises mouse immunoglobulin sequences only. A fully human antibody may be generated in a human being, in a transgenic animal having human immunoglobulin germline sequences, by phage display or other molecular biological methods.

“Hypervariable region” refers to the amino acid residues of an antibody that are responsible for antigen-binding. The hypervariable region comprises amino acid residues from a “complementarity determining region” or “CDR” (e.g. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain as measured by the Kabat numbering system (Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain (Chothia and Lesk (1987) J Mol. Biol. 196: 901-917). As used herein, the term “framework” or “FR” residues refers to those variable domain residues other than the hypervariable region residues defined herein as CDR residues. CDR and FR residues are determined according to the standard sequence definition of Kabat. Kabat et al. (1987) Sequences of Proteins of Immunological Interest, National Institutes of Health, Bethesda Md.

“Conservatively modified variants” or “conservative substitution” refers to substitutions of amino acids are known to those of skill in this art and may be made generally without altering the biological activity of the resulting molecule, even in essential regions of the polypeptide. Such exemplary substitutions are preferably made in accordance with those set forth in Table 1 as follows:

TABLE 1
Exemplary Conservative
Amino Acid Substitutions

| Original | Conservative |
|---|---|
| residue | substitution |
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

In addition, those of skill in this art recognize that, in general, single amino acid substitutions in non-essential regions of a polypeptide do not substantially alter biological activity. See, e.g., Watson et al. (1987) Molecular Biology of the Gene, The Benjamin/Cummings Pub. Co., p. 224 (4th Edition).

The phrase “consists essentially of” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J Mol. Biol. 222: 581-597, for example. See also Presta (2005) J. Allergy Clin. Immunol. 116:731.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Variable regions” or “V region” as used herein means the segment of IgG chains which is variable in sequence between different antibodies. It extends to Kabat residue 109 in the light chain and 113 in the heavy chain.

The term “buffer” encompasses those agents which maintain the solution pH of the formulations of the invention in an acceptable range, or, for lyophilized formulations of the invention, provide an acceptable solution pH prior to lyophilization.

The terms “lyophilization,” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage.

The term “pharmaceutical formulation” refers to preparations which are in such form as to permit the active ingredients to be effective, and which contains no additional components which are toxic to the subjects to which the formulation would be administered. The term “formulation” and “pharmaceutical formulations” are used interchangeably throughout.

“Pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

A “reconstituted” formulation is one that has been prepared by dissolving a lyophilized protein formulation in a diluent such that the protein is dispersed in the reconstituted formulation. The reconstituted formulation is suitable for administration, e.g. parenteral administration), and may optionally be suitable for subcutaneous administration.

“Reconstitution time” is the time that is required to rehydrate a lyophilized formulation with a solution to a particle-free clarified solution.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, in one embodiment, a stable formulation is a formulation with no significant changes observed at a refrigerated temperature (2-8° C.) for at least 12 months. In another embodiment, a stable formulation is a formulation with no significant changes observed at a refrigerated temperature (2-8° C.) for at least 18 months. In another embodiment, stable formulation is a formulation with no significant changes observed at room temperature (23-27° C.) for at least 3 months. In another embodiment, stable formulation is a formulation with no significant changes observed at room temperature (23-27° C.) for at least 6 months. In another embodiment, stable formulation is a formulation with no significant changes observed at room temperature (23-27° C.) for at least 12 months. In another embodiment, stable formulation is a formulation with no significant changes observed at room temperature (23-27° C.) for at least 18 months. The criteria for stability for an antibody formulation are as follows. Typically, no more than 10%, preferably 5%, of antibody monomer is degraded as measured by SEC-HPLC. Typically, the formulation is colorless, or clear to slightly opalescent by visual analysis. Typically, the concentration, pH and osmolality of the formulation have no more than +/−10% change. Potency is typically within 60-140%, preferably 80-120% of the control or reference. Typically, no more than 10%, preferably 5% of clipping of the antibody is observed, i.e., % low molecular weight species as determined, for example, by HP-SEC. Typically, no more than 10%, preferably no more than 5% of aggregation of the antibody is observed, i.e. % high molecular weight species as determined, for example, by HP-SEC.

An antibody “retains its physical stability” in a pharmaceutical formulation if it shows no significant increase of aggregation, precipitation and/or denaturation upon visual examination of color and/or clarity, or as measured by UV light scattering, size exclusion chromatography (SEC) and dynamic light scattering. The changes of protein conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical formulation, if the biological activity of the antibody at a given time is within a predetermined range of the biological activity exhibited at the time the pharmaceutical formulation was prepared. The biological activity of an antibody can be determined, for example, by an antigen binding assay. Formulations of the invention include antibodies and fragments thereof that are biologically active when reconstituted or in liquid form

The term “isotonic” means that the formulation of interest has essentially the same osmotic pressure as human blood. Isotonic formulations will generally have an osmotic pressure from about 270-328 mOsm. Slightly hypotonic pressure is 250-269 and slightly hypertonic pressure is 328-350 mOsm. Osmotic pressure can be measured, for example, using a vapor pressure or ice-freezing type osmometer.

A “non-reducing sugar” is a sugar not capable of acting as a reducing agent because it does not contain or cannot be converted to contain a free aldehyde group or a free ketone group. Examples of non-reducing sugars include but are not limited to dissacharrides such as sucrose and trehalose and trisaccharide sugars such as raffinose. In some embodiments of the invention, the non-reducing sugar is a disaccharide sugar. In some embodiments, the non-reducing sugar is a trisaccharide sugar.

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) (Merck Sharp & Dohme Corp., Whitehouse Station, N.J.) and which comprises the heavy and light chain amino acid sequences and CDRs described in Table 2. Pembrolizumab is approved by the U.S. FDA for the treatment of patients with unresectable or metastatic melanoma, for the adjuvant treatment of patients with melanoma with involvement of lymph node(s) following complete resection, and for the treatment of certain patients with recurrent or metastatic head and neck squamous cell cancer (HNSCC), classical Hodgkin lymphoma (cHL), urothelial carcinoma, gastric cancer, cervical cancer, primary mediastinal large-B-cell lymphoma, microsatellite instability-high (MSI-H) cancer, esophageal cancer, hepatocellular carcinoma, merkel cell carcinoma, renal cell carcinoma, endometrial carcinoma, small cell lung cancer, and non-small cell lung cancer, as described in the Prescribing Information for KEYTRUDA™ (Merck & Co., Inc., Whitehouse Station, N.J. USA; initial U.S. approval 2014, updated September 2019).

As used herein, a “pembrolizumab variant” refers to a derivative of a pembrolizumab antibody that substantially retains its biological activity of binding to antigen (i.e., human PD-1) and inhibiting its activity (e.g., blocking the binding of PD-1 to PD-L1 and/or PD-L2). In embodiments of the invention, the pembrolizumab variant comprises light chain and heavy chain sequences that are identical to those in pembrolizumab (SEQ ID NO:5 and 10, respectively), except for having up to 10, up to 9, up to 8, up to 7, up to 6, up to 5, up to 4, up to 3, up to 2, 1, 2, 3, 4, 5, 6, 7, 8, 9, or 10 conservative amino acid substitutions at amino acid positions that are located outside of the light chain CDRs and outside of the heavy chain CDRs, e.g., the variant positions are located in the framework regions or the constant region. In other words, in these embodiments, pembrolizumab and a pembrolizumab variant comprise identical CDR sequences, but differ from each other due to having a conservative amino acid substitution at no more than ten other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

### II. Formulations of the Invention

The formulations of the invention minimize the formation of antibody aggregates and particulates, high and low molecular weight species, minimize oxidation of methionine residues, and Met105 of pembrolizumab in particular, and ensure that the antibody retains biological activity over time.

The invention includes various formulations of a PD-1 antibody, or antigen binding fragment thereof, as described in more detail, infra. For example, the invention includes formulations comprising (i) an anti-PD-1 antibody or antigen binding fragment thereof, (ii) a buffer (e.g., histidine or acetate), (iii) a stabilizer (e.g., a non-reducing sugar such as sucrose or trehalose, or sorbitol, mannitol, (2-hydroxypropyl)-β-cyclodextrin, arginine, proline, histidine or glycine); (iv) a non-ionic surfactant (e.g., polysorbate 20, a poloxamer, vitamin E D-α-tocopherol polyethylene glycol succinate (TPGS), polyethylene glycol tert-octylphenyl ether (trade name: TRITON™ X-100), octyl maltoside (OM), and dodecyl maltoside (DDM), or an ionic surfactant (e.g. dimethyl-dodecylamine oxide (DDAO) and SDS); and (v) an antioxidant (e.g., methionine). In further embodiments, the formulations of the invention comprise a viscosity-reducer (e.g. arginine, proline, histidine or pharmaceutically acceptable salts thereof) and/or a metal chelator (e.g. DTPA).

### Anti-PD-1 Antibodies and Antigen-Binding Fragments Thereof

The invention provides stable biological formulations comprising antibodies or antigen binding fragments thereof, which specifically bind to human PD-1 (e.g. a human or humanized anti-PD-1 antibody) as the active pharmaceutical ingredient (API), as well as methods for using the formulations of the invention. Any anti-PD-1 antibody or antigen binding fragment thereof can be used in the formulations and methods of the invention. In particular embodiments, the API is an anti-PD-1 antibody, which is selected from pembrolizumab and nivolumab. In specific embodiments, the anti-PD-1 antibody is pembrolizumab. In alternative embodiments, the anti-PD-1 antibody is nivolumab. Table 2 provides amino acid sequences for exemplary anti-human PD-1 antibodies pembrolizumab and nivolumab. Alternative PD-1 antibodies and antigen-binding fragments that are useful in the formulations and methods of the invention are shown in Table 3.

In some embodiments, an anti-human PD-1 antibody or antigen binding fragment thereof for use in the formulations of the invention comprises three light chain CDRs of CDRL1, CDRL2 and CDRL3 and/or three heavy chain CDRs of CDRH1, CDRH2 and CDRH3.

In one embodiment of the invention, CDRL1 is SEQ ID NO:1 or a variant of SEQ ID NO:1, CDRL2 is SEQ ID NO:2 or a variant of SEQ ID NO:2, and CDRL3 is SEQ ID NO:3 or a variant of SEQ ID NO:3.

In one embodiment, CDRH1 is SEQ ID NO:6 or a variant of SEQ ID NO:6, CDRH2 is SEQ ID NO: 7 or a variant of SEQ ID NO:7, and CDRH3 is SEQ ID NO:8 or a variant of SEQ ID NO:8.

In one embodiment, the three light chain CDRs are SEQ ID NO:1, SEQ ID NO:2, and SEQ ID NO:3 and the three heavy chain CDRs are SEQ ID NO:6, SEQ ID NO:7 and SEQ ID NO:8.

In an alternative embodiment of the invention, CDRL1 is SEQ ID NO:11 or a variant of SEQ ID NO:11, CDRL2 is SEQ ID NO:12 or a variant of SEQ ID NO:12, and CDRL3 is SEQ ID NO:13 or a variant of SEQ ID NO:13.

In one embodiment, CDRH1 is SEQ ID NO:16 or a variant of SEQ ID NO:16, CDRH2 is SEQ ID NO:17 or a variant of SEQ ID NO:17, and CDRH3 is SEQ ID NO:18 or a variant of SEQ ID NO:18.

In one embodiment, the three light chain CDRs are SEQ ID NO:1, SEQ ID NO:2, and SEQ ID NO:3 and the three heavy chain CDRs are SEQ ID NO:6, SEQ ID NO:7 and SEQ ID NO:8.

In an alternative embodiment, the three light chain CDRs are SEQ ID NO:11, SEQ ID NO:12, and SEQ ID NO:13 and the three heavy chain CDRs are SEQ ID NO:16, SEQ ID NO:17 and SEQ ID NO:18.

In a further embodiment of the invention, CDRL1 is SEQ ID NO:21 or a variant of SEQ ID NO:21, CDRL2 is SEQ ID NO:22 or a variant of SEQ ID NO:22, and CDRL3 is SEQ ID NO:23 or a variant of SEQ ID NO:23.

In yet another embodiment, CDRH1 is SEQ ID NO:24 or a variant of SEQ ID NO:24, CDRH2 is SEQ ID NO: 25 or a variant of SEQ ID NO:25, and CDRH3 is SEQ ID NO:26 or a variant of SEQ ID NO:26.

In another embodiment, the three light chain CDRs are SEQ ID NO:21, SEQ ID NO:22, and SEQ ID NO:23 and the three heavy chain CDRs are SEQ ID NO:24, SEQ ID NO:25 and SEQ ID NO:26.

Some antibody and antigen binding fragments of the formulations of the invention comprise a light chain variable region and a heavy chain variable region. In some embodiments, the light chain variable region comprises SEQ ID NO:4 or a variant of SEQ ID NO:4, and the heavy chain variable region comprises SEQ ID NO:9 or a variant of SEQ ID NO:9. In further embodiments, the light chain variable region comprises SEQ ID NO:14 or a variant of SEQ ID NO:14, and the heavy chain variable region comprises SEQ ID NO:19 or a variant of SEQ ID NO:19. In further embodiments, the heavy chain variable region comprises SEQ ID NO:27 or a variant of SEQ ID NO:27 and the light chain variable region comprises SEQ ID NO:28 or a variant of SEQ ID NO:28, SEQ ID NO:29 or a variant of SEQ ID NO:29, or SEQ ID NO:30 or a variant of SEQ ID NO:30. In such embodiments, a variant light chain or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In one embodiment of the formulations of the invention, the antibody or antigen binding fragment comprises a light chain variable region comprising or consisting of SEQ ID NO:4 and a heavy chain variable region comprising or consisting SEQ ID NO:9. In a further embodiment, the antibody or antigen binding fragment comprises a light chain variable region comprising or consisting of SEQ ID NO:14 and a heavy chain variable region comprising or consisting of SEQ ID NO:19. In one embodiment of the formulations of the invention, the antibody or antigen binding fragment comprises a light chain variable region comprising or consisting of SEQ ID NO:28 and a heavy chain variable region comprising or consisting SEQ ID NO:27. In a further embodiment, the antibody or antigen binding fragment comprises a light chain variable region comprising or consisting of SEQ ID NO:29 and a heavy chain variable region comprising or consisting SEQ ID NO:27. In another embodiment, the antibody or antigen binding fragment comprises a light chain variable region comprising or consisting of SEQ ID NO:30 and a heavy chain variable region comprising or consisting SEQ ID NO:27.

In another embodiment, the formulations of the invention comprise an antibody or antigen binding protein that has a VL domain and/or a VH domain with at least 95%, 90%, 85%, 80%, 75% or 50% sequence homology to one of the VL domains or VH domains described above, and exhibits specific binding to PD-1. In another embodiment, the antibody or antigen binding protein of the formulations of the invention comprises VL and VH domains having up to 1, 2, 3, 4, or 5 or more amino acid substitutions, and exhibits specific binding to PD-1.

In any of the embodiments above, the API may be a full-length anti-PD-1 antibody or an antigen binding fragment thereof that specifically binds human PD-1. In certain embodiments, the API is a full-length anti-PD-1 antibody selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Different constant domains may be appended to the VL and VH regions provided herein. For example, if a particular intended use of an antibody (or fragment) of the invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

In embodiments of the invention, the API is an anti-PD-1 antibody comprising a light chain comprising or consisting of a sequence of amino acid residues as set forth in SEQ ID NO:5 and a heavy chain comprising or consisting of a sequence of amino acid residues as set forth in SEQ ID NO:10. In alternative embodiments, the API is an anti-PD-1 antibody comprising a light chain comprising or consisting of a sequence of amino acid residues as set forth in SEQ ID NO:15 and a heavy chain comprising or consisting of a sequence of amino acid residues as set forth in SEQ ID NO:20. In further embodiments, the API is an anti-PD-1 antibody comprising a light chain comprising or consisting of a sequence of amino acid residues as set forth in SEQ ID NO:32 and a heavy chain comprising or consisting of a sequence of amino acid residues as set forth in SEQ ID NO:31. In additional embodiments, the API is an anti-PD-1 antibody comprising a light chain comprising or consisting of a sequence of amino acid residues as set forth in SEQ ID NO:33 and a heavy chain comprising or consisting of a sequence of amino acid residues as set forth in SEQ ID NO:31. In yet additional embodiments, the API is an anti-PD-1 antibody comprising a light chain comprising or consisting of a sequence of amino acid residues as set forth in SEQ ID NO:34 and a heavy chain comprising or consisting of a sequence of amino acid residues as set forth in SEQ ID NO:31. In some formulations of the invention, the API is pembrolizumab, a pembrolizumab variant or a pembrolizumab biosimilar. In some formulations of the invention, the API is nivolumab, a nivolumab variant or a nivolumab biosimilar.

Ordinarily, amino acid sequence variants of the anti-PD-1 antibodies and antigen binding fragments of the invention will have an amino acid sequence having at least 75% amino acid sequence identity with the amino acid sequence of a reference antibody or antigen binding fragment (e.g. heavy chain, light chain, VH, VL, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the anti-PD-1 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

Likewise, either class of light chain can be used in the compositions and methods herein. Specifically, kappa, lambda, or variants thereof are useful in the present compositions and methods.

TABLE 2
Exemplary PD-1 Antibody Sequences

| | | |
|---|---|---|
| | | SEQ |
| Antibody | | ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSY | 4 |
| Region | LHWYQQKPGQAPRLLIYLASYLESGVPARFSGSGSG | |
| | TDFTLTISSLEPEDFAVYYCQHSRDLPLTFGGGTKV | |
| | EIK | |
| Light | EIVLTQSPATLSLSPGERATLSCRASKGVSTSGYSY | 5 |
| Chain | LHWYQQKPGQAPRLLIYLASYLESGVPARFSGSGSG | |
| | TDFTLTISSLEPEDFAVYYCQHSRDLPLTFGGGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYW | 9 |
| Region | VRQAPGQGLEWMGGINPSNGGTNFNEKFKNRVTLTT | |
| | DSSTTTAYMELKSLQFDDTAVYYCARRDYRFDMGFD | |
| | YWGQGTTVTVSS | |
| Heavy | QVQLVQSGVEVKKPGASVKVSCKASGYTFTNYYMYW | 10 |
| Chain | VRQAPGQGLEWMGGINPSNGGTNFNEKFKNRVTLTT | |
| | DSSTTTAYMELKSLQFDDTAVYYCARRDYRFDMGFD | |
| | YWGQGTTVTVSSASTKGPSVFPLAPCSRSTSESTAA | |
| | LGCLVKDYFPEPVTVSWNSGALTSGVHTFPAVLQSS | |
| | GLYSLSSVVTVPSSSLGTKTYTCNVDHKPSNTKVDK | |
| | RVESKYGPPCPPCPAPEFLGGPSVFLFPPKPKDTLM | |
| | ISRTPEVTCVVVDVSQEDPEVQFNWYVDGVEVHNAK | |
| | TKPREEQFNSTYRVVSVLTVLHQDWLNGKEYKCKVS | |
| | NKGLPSSIEKTISKAKGQPREPQVYTLPPSQEEMTK | |
| | NQVSLTCLVKGFYPSDIAVEWESNGQPENNYKTTPP | |
| | VLDSDGSFFLYSRLTVDKSRWQEGNVFSCSVMHEAL | |
| | HNHYTQKSLSLSLGK | |

Nivolumab Light Chain

| | | |
|---|---|---|
| CDR1 | RASQSVSSYLA | 11 |
| CDR2 | DASNRAT | 12 |
| CDR3 | QQSSNWPRT | 13 |
| Variable | EIVLTQSPATLSLSPGERATLSCRASQSVSSYLAWY | 14 |
| Region | QQKPGQAPRLLIYDASNRATGIPARFSGSGSGTDFT | |
| | LTISSLEPEDFAVYYCQQSSNWPRTFGQGTKVEIK | |
| Light | EIVLTQSPATLSLSPGERATLSCRASQSVSSYLAWY | 15 |
| Chain | QQKPGQAPRLLIYDASNRATGIPARFSGSGSGTDFT | |
| | LTISSLEPEDFAVYYCQQSSNWPRTFGQGTKVEIKR | |
| | TVAAPSVFIFPPSDEQLKSGTASVVCLLNNFYPREA | |
| | KVQWKVDNALQSGNSQESVTEQDSKDSTYSLSSTLT | |
| | LSKADYEKHKVYACEVTHQGLSSPVTKSFNRGEC | |

Nivolumab Heavy Chain

| | | |
|---|---|---|
| CDR1 | NSGMH | 16 |
| CDR2 | VIWYDGSKRYYADSVKG | 17 |
| CDR3 | NDDY | 18 |
| Variable | QVQLVESGGGVVQPGRSLRLDCKASGITFSNSGMHW | 19 |
| Region | VRQAPGKGLEWVAVIWYDGSKRYYADSVKGRFTISR | |
| | DNSKNTLFLQMNSLRAEDTAVYYCATNDDYWGQGTL | |
| | VTVSS | |
| Heavy | QVQLVESGGGVVQPGRSLRLDCKASGITFSNSGMHW | 20 |
| Chain | VRQAPGKGLEWVAVIWYDGSKRYYADSVKGRFTISR | |
| | DNSKNTLFLQMNSLRAEDTAVYYCATNDDYWGQGTL | |
| | VTVSSASTKGPSVFPLAPCSRSTSESTAALGCLVKD | |
| | YFPEPVTVSWNSGALTSGVHTFPAVLQSSGLYSLSS | |
| | VVTVPSSSLGTKTYTCNVDHKPSNTKVDKRVESKYG | |
| | PPCPPCPAPEFLGGPSVFLFPPKPKDTLMISRTPEV | |
| | TCVVVDVSQEDPEVQFNWYVDGVEVHNAKTKPREEQ | |
| | FNSTYRVVSVLTVLHQDWLNGKEYKCKVSNKGLPSS | |
| | IEKTISKAKGQPREPQVYTLPPSQEEMTKNQVSLTC | |
| | LVKGFYPSDIAVEWESNGQPENNYKTTPPVLDSDGS | |
| | FFLYSRLTVDKSRWQEGNVFSCSVMHEALHNHYTQK | |
| | SLSLSLGK | |

TABLE 3
Additional PD-1 Antibodies and Antigen
Binding Fragments Useful in the Formulations,
Methods and Uses of the Invention.

| A. Antibodies and antigen binding fragments | |
|---|---|
| comprising light and heavy chain CDRs of | |
| hPD-1.08A in WO2008/156712 | |
| CDRL1 | SEQ ID NO: 21 |
| CDRL2 | SEQ ID NO: 22 |
| CDRL3 | SEQ ID NO: 23 |
| CDRH1 | SEQ ID NO: 24 |
| CDRH2 | SEQ ID NO: 25 |
| CDRH3 | SEQ ID NO: 26 |
| C. Antibodies and antigen binding fragments | |
| comprising the mature h109A heavy chain | |
| variable region and one of the mature K09A | |
| light chain variable regions in WO2008/156712 | |
| Heavy chain VR | SEQ ID NO: 27 |
| Light chain VR | SEQ ID NO: 28, SEQ ID NO: 29, |
| | SEQ ID NO: 30 |
| D. Antibodies and antigen binding fragments | |
| comprising the mature 409 heavy chain and | |
| one of the mature KO9A light chains in | |
| WO2008/156712 | |
| Heavy chain | SEQ ID NO: 31 |
| Light chain | SEQ ID NO: 32, SEQ ID NO: 33, |
| | SEQ ID NO: 34 |

In some embodiments of the formulations of the invention, the API (i.e. the anti-PD-1 antibody or antigen binding fragment thereof) is present in a concentration of about 25 mg/mL to about 275 mg/mL. In additional embodiments, the API is present in a concentration of from about 5 mg/mL to about 25 mg/mL. In some embodiments of the formulations of the invention, the API is present in a concentration of about 5 mg/mL to about 200 mg/mL. In alternative embodiments, the API is present in a concentration of about 5 mg/mL, about 10 mg/mL, about 25 mg/mL, about 50 mg/mL, about 75 mg/mL, about 100 mg/mL, about 125 mg/mL, about 130 mg/mL about 150 mg/mL, about 165 mg/mL, about 167 mg/mL about 175 mg/mL, about 200 mg/mL, about 210 mg/mL, about 225 mg/mL, about 230 mg/mL, about 240 mg/mL, about 250 mg/mL, about 260 mg/mL, about 270 mg/mL, or about 275 mg/mL.

In one embodiment, the API is present in a concentration of about 165 to about 170 mg/mL.

In one embodiment, the API is present in a concentration of about 167 mg/mL.

In one embodiment, the API is present in a concentration of about 130 mg/mL.

In one embodiment, the API is present in a concentration of about 200 mg/mL.

In one embodiment, the API is present in a concentration of about 225 mg/mL.

In one embodiment, the API is present in a concentration of about 250 mg/mL.

In one embodiment, the API is present in a concentration greater than 200 mg/mL and less than or equal to 250 mg/mL.

In additional embodiments, the API is present in a concentration of from about 5 mg/mL to about 75 mg/mL, from about 50 mg/mL to about 200 mg/mL; from about 75 mg/mL to about 200 mg/mL; from about 100 mg/mL to about 200 mg/mL; from about 25 mg/mL to about 175 mg/mL; from about 50 mg/mL to about 175 mg/mL; from about 75 mg/mL to about 175 mg/mL; from about 100 mg/mL to about 175 mg/mL; from about 25 mg/mL to about 150 mg/mL; from about 50 mg/mL to about 150 mg/mL; from about 75 mg/mL to about 150 mg/mL; from about 100 mg/mL to about 150 mg/mL; from about 25 mg/mL to about 125 mg/mL; from about 50 mg/mL to about 125 mg/mL; from about 75 mg/mL to about 125 mg/mL; from about 25 mg/mL to about 100 mg/mL, from about 125 mg/mL to about 175 mg/mL, from about 125 mg/mL to about 200 mg/mL, from about 5 mg/mL to 200 mg/mL, from about 200 mg/mL to 250 mg/mL, from about 5 mg/mL to 250 mg/mL, from about 75 mg/mL to 250 mg/mL, from about 100 mg/mL to 250 mg/mL, or from about 150 mg/mL to 250 mg/mL.

### Formulation Excipients

The formulations of the invention comprise at least one excipient that stabilizes the formulation. In some embodiments, the formulation comprises more than one stabilizer.

In some embodiments of the formulations of the invention, the stabilizer is a non-reducing sugar. In further embodiments, the non-reducing sugar is sucrose. In additional embodiments, the non-reducing sugar is trehalose. In other embodiments, the non-reducing sugar is raffinose. In specific embodiments, the non-reducing sugar is a non-reducing disaccharide sugar (e.g., sucrose and trehalose).

In some embodiments, the anti-human PD-1 antibody formulations of the invention comprise about 1.5 to about 8.0% weight/volume (w/v) stabilizer selected from the group consisting of: a non-reducing sugar, (2-hydroxypropyl)-β-cyclodextrin, mannitol, sorbitol, L-arginine, a pharmaceutically acceptable salt of L-arginine, L-proline, a pharmaceutically acceptable salt of L-proline, L-histidine, a pharmaceutically acceptable salt of L-histidine, glycine, and a pharmaceutically acceptable salt of glycine.

In some embodiments, the anti-human PD-1 antibody formulations of the invention comprise about 1.5% to about 8.0% weight/volume (w/v) stabilizer selected from the group consisting of: sucrose, trehalose, (2-hydroxypropyl)-β-cyclodextrin, mannitol, sorbitol, L-arginine, a pharmaceutically acceptable salt of L-arginine, L-proline, a pharmaceutically acceptable salt of L-proline, L-histidine, a pharmaceutically acceptable salt of L-histidine, glycine, and a pharmaceutically acceptable salt of glycine.

In some embodiments, the anti-human PD-1 antibody formulations of the invention comprise a stabilizer selected from the group consisting of: about 6% to about 8% w/v sucrose, trehalose or (2-hydroxypropyl)-β-cyclodextrin; about 3% to about 5% w/v mannitol, sorbitol, L-arginine, or a pharmaceutically acceptable salt of L-arginine, or L-proline, or a pharmaceutically acceptable salt of L-proline; and about 1.8 to about 2.2% w/v glycine, or a pharmaceutically acceptable salt thereof about 1.5% to 1.9% w/v L-proline, or a pharmaceutically acceptable sale of L-proline; about 1.9%-3.3% w/v L-arginine, or a pharmaceutically acceptable salt of L-arginine; and about 2% to about 3% L-histidine, or a pharmaceutically acceptable salt of L-histidine.

In some embodiments, the stabilizer is about 6% to about 8% w/v non-reducing sugar.

In some embodiments, the stabilizer is about 6% to about 8% w/v non-reducing disaccharide sugar.

In some embodiments, the stabilizer is about 6% to about 8% w/v sucrose.

In some embodiments, the stabilizer is about 6% to about 8% w/v trehalose.

In some embodiments, the stabilizer is about 6% to about 8% w/v (2-hydroxypropyl)-β-cyclodextrin.

In some embodiments, the stabilizer is sucrose, trehalose or (2-hydroxypropyl)-O-cyclodextrin, which is present in an amount of about 6% to about 8% w/v. In further embodiments, the sucrose, trehalose or (2-hydroxypropyl)-β-cyclodextrin is present in an amount of about 6.5% to about 7.5% w/v. In still further embodiments, the sucrose, trehalose or (2-hydroxypropyl)-β-cyclodextrin is present in an amount of about 6% w/v, about 6.25% w/v, about 6.5% w/v, about 6.75% w/v, about 7% w/v, about 7.25% w/v, about 7.5% w/v, about 7.75% w/v or about 8% w/v.

In some embodiments, the stabilizer is about 3% to about 5% w/v mannitol.

In some embodiments, the stabilizer is about 3% to about 5% w/v sorbitol.

In some embodiments, the stabilizer is about 3% to about 5% w/v L-arginine, or a pharmaceutically acceptable salt thereof. In some embodiments, the stabilizer is about 1.9%-3.3% w/v L-arginine, or a pharmaceutically acceptable salt of L-arginine. For example, the stabilizer can be about 2.1% w/v L-arginine, or a pharmaceutically acceptable salt of L-arginine. In another example, the stabilizer can be about 3.1% w/v L-arginine, or a pharmaceutically acceptable salt of L-arginine. The pharmaceutically acceptable salt can be L-arginine hydrochloride (i.e., L-arginine HCl).

In certain embodiments, the formulation of the invention comprises arginine as stabilizer, e.g., L-arginine or a pharmaceutically acceptable salt thereof. In additional embodiments, the formulations of the invention comprise arginine hydrochloride (i.e. arginine HCl). In further embodiments, the formulations comprise arginine succinate. In further embodiments, the arginine is L-arginine.

In some embodiments, the stabilizer is about 3% to about 5% w/v proline, e.g., L-proline, or a pharmaceutically acceptable salt thereof. In some embodiments, the stabilizer is about 1.5% to 1.9% w/v L-proline, or a pharmaceutically acceptable sale of L-proline. In additional embodiments, the formulations of the invention comprise proline hydrochloride (i.e., proline HCl). In further embodiments, the formulations comprise L-proline.

In some embodiments, the stabilizer is about 2% to about 3% L-histidine, or a pharmaceutically acceptable salt of L-histidine. For example, the stabilizer can be about 2.3% to 2.5% w/v L-histidine, or a pharmaceutically acceptable salt of L-histidine. The pharmaceutically acceptable salt can be L-histidine hydrochloride (e.g., L-histidine HCl).

In some embodiments, the stabilizer is mannitol, sorbitol, L-arginine, a pharmaceutically acceptable salt of L-arginine, L-proline, or a pharmaceutically acceptable salt of L-proline, which is present in an amount of about 3% to about 5% w/v. In further embodiments, the mannitol, sorbitol, L-arginine, pharmaceutically acceptable salt of L-arginine, L-proline, or a pharmaceutically acceptable salt of L-proline, is present in an amount of about 3.5% to about 4.5% w/v. In still further embodiments, the mannitol, sorbitol, L-arginine, pharmaceutically acceptable salt of L-arginine, L-proline, or pharmaceutically acceptable salt of L-proline, is present in an amount of about 3% w/v, about 3.25% w/v, about 3.5% w/v, about 3.75% w/v, about 4% w/v, about 4.25% w/v, about 4.5% w/v, about 4.75% w/v, or about 5% w/v.

In some embodiments, the stabilizer is about 1.8 to about 2.2% w/v glycine, or a pharmaceutically acceptable salt thereof.

In certain embodiments, the formulation of the invention comprises glycine or a pharmaceutically acceptable salt thereof. In additional embodiments, the formulations of the invention comprise sodium glycinate.

In specific embodiments, the stabilizer is glycine, which is present in an amount of about 150 mM to about 200 mM, or about 150 mM, about 160 mM, about 170 mM, about 175 mM, about 180 mM, about 190 mM or about 200 mM.

In certain embodiments, the stabilizer is glycine, which is present in an amount of about 1.8 to about 2.2% w/v, about 1.5 to about 2.5%, or about 1.8 to about 2.5% or about 1.5 to about 2.2%. In specific embodiments, the glycine is present in an amount of about 1.8%, about 2.0% about 2.2%, or about 2.5%.

In some embodiments, the anti-human PD-1 antibody formulations of the invention comprise a stabilizer selected from the group consisting of (1) about 6% to about 8% w/v sucrose, trehalose or (2-hydroxypropyl)-β-cyclodextrin; (2) about 3% to about 5% mannitol, sorbitol, L-proline, or a pharmaceutically acceptable salt of L-proline; and (3) about 1.8 to about 2.2% w/v glycine, or a pharmaceutically acceptable salt thereof.

In some embodiments, the formulations of the invention comprise arginine, e.g., L-arginine, or a pharmaceutically acceptable salt thereof, which may provide additional stability to the formulation, as well as control viscosity, which allows formulation at high API concentration. In certain embodiments of the invention, alternate or additional viscosity reducing agents are included. In particular embodiments, the formulation comprises a viscosity reducing agent which is selected from: (a) L-arginine, or a pharmaceutically acceptable thereof, (b) L-lysine, or a pharmaceutically acceptable thereof, (c) L-histidine, or a pharmaceutically acceptable thereof, (d) L-glutamine, or a pharmaceutically acceptable thereof, and (e) a mixture two or more viscosity reducing agents specified in a)-d).

As noted above, in specific embodiments, the formulations of the invention comprise a high concentration of API (e.g. about 75 mg/mL to about 250 mg/mL or about 200 mg/mL to about 250 mg/mL). In particular embodiments wherein a high concentration of API is employed, the formulations of the invention also comprise arginine, e.g., L-arginine, or a pharmaceutically acceptable salt thereof, e.g. an amount of L-arginine from about 0.25% to about 3.0% w/v.

In some embodiments of the invention, the formulation has a viscosity of ≤60 centipoise at 20° C. In some embodiments, the formulation has a viscosity of ≤100 cP, ≤90 cP, ≤80 cP, ≤70 cP, ≤75 cP, ≤60 cP, or ≤50 cP at 20° C.

In some embodiments of the invention, the viscosity reducing agent (e.g. L-arginine or pharmaceutically acceptable salt) is present in the formulations in an amount of 0.25% to about 3% weight/volume. In additional embodiments, the viscosity reducing agent (L-arginine or pharmaceutically acceptable salt) is present in an amount of about 0.25% w/v, about 0.50% w/v, about 0.75% w/v, about 1.0% w/v, about 1.25% w/v, about 1.5% w/v, about 1.75% w/v, about 2.0% w/v, about 2.25% w/v, about 2.5% w/v, about 2.75% w/v or about 3.0% w/v. In further embodiments, the viscosity reducing agent is present in an amount of about 0 to about 2.75% w/v, 0 to about 2.5% w/v, 0 to about 2.25% w/v, 0 to about 2% w/v, 0 to about 1.75% w/v, 0 to about 1.5% w/v, 0 to about 1.25% w/v, 0 to about 1.0% w/v, about 0.5% to about 3.0% w/v, about 0.5% to about 2.75% w/v, about 0.5% to about 2.5% w/v, about 0.5% to about 2.25% w/v, about 0.5% to about 2% w/v, about 0.5% to about 1.75% w/v, about 0.5% to about 1.5% w/v, about 0.5% to about 1.25% w/v, about 0.5% to about 1.0% w/v, about 1.0% to about 3.0% w/v, about 1.0% to about 2.75% w/v, about 1.0% to about 2.5% w/v, about 1.0% to about 2.25% w/v, about 1.0% to about 2% w/v, about 1.0% to about 1.75% w/v, about 1.0% to about 1.5% w/v, about 1.5% to about 3.0% w/v, about 1.5% to about 2.75% w/v, about 1.5% to about 2.5% w/v, about 1.5% to about 2.25% w/v, about 1.5% to about 2% w/v, or about 2% to about 3% w/v.

In some embodiments of the invention, the formulation comprises about 1.8 to about 8.0% weight/volume (w/v) stabilizer, which is selected from the group consisting of: a non-reducing sugar, (2-hydroxypropyl)β-cyclodextrin, mannitol, sorbitol, L-arginine, a pharmaceutically acceptable salt of L-arginine, L-proline, a pharmaceutically acceptable salt of L-proline, glycine, and a pharmaceutically acceptable salt of glycine;

In some embodiments of the invention, the stabilizer is selected from the group consisting of: about 6% to about 8% w/v sucrose, trehalose or (2-hydroxypropyl)β-cyclodextrin; about 3% to about 5% w/v mannitol, sorbitol, or proline, or a pharmaceutically acceptable salt thereof; and about 1.8 to about 2.2% w/v glycine, or a pharmaceutically acceptable salt thereof, and the formulation further comprises L-arginine or a pharmaceutically acceptable salt thereof, which can be added in any of the amounts above to reduce viscosity of the formulation, especially when the API is present in high concentration (e.g. 75 mg/mL-275 mg/mL). It is understood that although L-arginine, or pharmaceutically acceptable salt thereof, may be added to reduce viscosity in such embodiments, the L-arginine or pharmaceutically acceptable salt, may also be serving to stabilize the formulation and may impart additional stability relative to the formulation without L-arginine or pharmaceutically acceptable salt.

In addition to an anti-PD-1 antibody or antigen binding fragment thereof, and a stabilizer in the amounts/concentrations specified above, the formulations of the invention also comprise a buffer. In some embodiments the buffer is present in an amount of about 5 mM to about 20 mM, which provides for a pH in the range of about 4.5 to 6.4.

In some embodiments of the invention, the buffer provides the formulation a pH in the range from about 4.5 to about 6.5. In further embodiments, the buffer has a pH in a range of about 5.0 to about 6.0. In still further embodiments, the pH is from about 5.3 to about 5.8. In other embodiments, the pH is from about 6.0 to about 6.4.

In particular embodiments, the buffer has a pH of about 5.0, about 5.1, about 5.2, about 5.3, about 5.4, about 5.5, about 5.6, about 5.7, about 5.8, about 5.9, about 6.0, about 6.2 or about 6.4. Examples of buffers that will control the pH in this range include succinate (sodium or potassium), histidine, sodium acetate, phosphate (sodium or potassium), Tris (tris (hydroxymethyl) aminomethane), diethanolamine, citrate (sodium) and other organic acid buffers.

In specific embodiments of the invention, the buffer is histidine or acetate at a pH of about 5.0 to about 6.0. In some embodiments, the buffer is an L-histidine buffer. In some preferred embodiments, the buffer is acetate. In embodiments where the formulation is lyophilized, it is preferred that the buffer is not acetate because acetate buffer systems are not compatible with the lyophilization process.

When a range of pH values is recited, such as “a pH between pH 5.5 and 6.0,” the range is intended to be inclusive of the recited values. Unless otherwise indicated, for lyophilized formula the pH refers to the pH after reconstitution of the lyophilized formulations of the invention. The pH is typically measured at 25° C. using standard glass bulb pH meter. As used herein, a solution comprising “histidine buffer at pH X” refers to a solution at pH X and comprising the histidine buffer, i.e. the pH is intended to refer to the pH of the solution.

In addition to an anti-PD-1 antibody or antigen binding fragment thereof, a stabilizer, and a buffer in the amounts/concentrations specified above, the formulations of the invention also comprise an anti-oxidant. In embodiments of the invention, the anti-oxidant is methionine. In embodiments of the invention, the anti-oxidant is L-methionine, or a pharmaceutically acceptable salt thereof. In further embodiments, the methionine is L-methionine. In other embodiments, the anti-oxidants is L-methionine HCl. In other embodiments, the anti-oxidant is histidine.

In some embodiments, the anti-oxidant (e.g. L-methionine) is present in the formulations of the invention in an amount of amount 1 mM to about 30 mM. In further embodiments, the anti-oxidant is present in an amount of about 5 mM to about 20 mM, about 5 mM to about 15 mM, about 5 mM to about 10 mM. In additional embodiments, the anti-oxidant is present in an amount of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM about 20 mM, about 22 mM, about 25 mM, about 28 mM, or about 30 mM.

In embodiments wherein the anti-oxidant is histidine, the histidine can be present in amounts up to 100 mM. In such embodiments, histidine can serve as a buffer and as an anti-oxidant in the formulations of the invention.

In addition to an anti-PD-1 antibody or antigen binding fragment thereof, a stabilizer, a buffer, and an anti-oxidant in the amounts/concentrations specified above, the formulations of the invention also comprise a surfactant. Surfactants are typically added to formulations to provide stability, reduce and/or prevent aggregation or to prevent and/or inhibit protein damage during processing conditions such as purification, filtration, freeze-drying, transportation, storage, and delivery. In some embodiments of the invention, a surfactant is useful for providing additional stability to the active ingredient(s), i.e. the anti-PD-1 antibody or antigen binding fragment thereof.

Surfactants that may be useful in the formulations of the invention include nonionic surfactants and ionic surfactants. In some embodiments, the surfactant is an ionic surfactant, which is present at a concentration of about 0.23% w/v to about 1.15% w/v. In particular embodiments, the concentration of ionic surfactant is about 0.23% w/v, about 0.25% w/v, about 0.30% w/v, about 0.35% w/v, about 0.40% w/v, about 0.45% w/v, about 0.50% w/v, about 0.55% w/v, about 0.60% w/v, about 0.65% w/v, about 0.70% w/v, about 0.75% w/v, about 0.80% w/v, about 0.85% w/v, about 0.90% w/v, about 0.95% w/v, about 1.0% w/v, about 1.05% w/v, about 1.10% w/v, about 1.15% w/v.

In some embodiments, the ionic surfactant is SDS.

In some embodiments, the non-ionic surfactant is dimethyl-dodecylamine oxide (DDAO). In some embodiments, the DDAO is present in a range of about 0.005% w/v to about 0.20% w/v.

In some embodiments, the surfactant is a non-ionic surfactant, which is present at a concentration of about 0.005% w/v to about 0.60% w/v. In particular embodiments, the non-ionic surfactant is present at a concentration of about 0.01% w/v, 0.02% w/v, 0.03% w/v, 0.04% w/v, 0.05% w/v, 0.06% w/v, 0.07% w/v, 0.08% w/v, 0.09% w/v, 0.10% w/v, 0.11% w/v, 0.12% w/v, 0.13% w/v, 0.14% w/v, 0.15% w/v, 0.16% w/v, 0.17% w/v, 0.18% w/v, 0.19% w/v, 0.20% w/v, 0.21% w/v, 0.22% w/v, 0.23% w/v, 0.24% w/v, 0.25% w/v, 0.26% w/v, 0.27% w/v, 0.28% w/v, 0.29% w/v, 0.30% w/v, 0.31% w/v, 0.32% w/v, 0.33% w/v, 0.34% w/v, 0.35% w/v, 0.36% w/v, 0.37% w/v, 0.38% w/v, 0.39% w/v, 0.40% w/v, 0.41% w/v, 0.42% w/v, 0.43% w/v, 0.44% w/v, 0.45% w/v, 0.46% w/v, 0.47% w/v, 0.48% w/v, 0.49% w/v, 0.5% w/v, 0.51% w/v, 0.52% w/v, 0.53% w/v, 0.54% w/v, 0.55% w/v, 0.56% w/v, 0.57% w/v, 0.58% w/v, 0.59% w/v, or 0.60% w/v.

In some embodiments, the non-ionic surfactant is polysorbate 20.

In some embodiments, the non-ionic surfactant is a poloxamer.

In some embodiments, the non-ionic surfactant is poloxamer 188 (P188).

In some embodiments, the non-ionic surfactant is poloxamer 388 (P388).

In some embodiments, the non-ionic surfactant is poloxamer 407 (P407).

In some embodiments, the non-ionic surfactant is vitamin E D-α-tocopherol polyethylene glycol succinate (TPGS).

In some embodiments, the non-ionic surfactant is polyethylene glycol tert-octylphenyl ether (tradename: TRITON™ X-100).

In some embodiments, the non-ionic surfactant is n-octyl β-D-maltoside (OM).

In some embodiments, the non-ionic surfactant is n-dodecyl β-D-maltoside (DDM).

The amount of surfactant to be included in the formulations of the invention is an amount sufficient to perform the desired function, i.e. a minimal amount necessary to stabilize the active pharmaceutical ingredient (i.e. the anti-PD-1 antibody or antigen binding fragment thereof) in the formulation. Typically, the surfactant is present in a concentration of from about 0.008% to about 0.6% w/v. In some embodiments of this aspect of the invention, the surfactant is present in the formulation in an amount from about 0.01% to about 0.04%; from about 0.01% to about 0.03%, from about 0.01% to about 0.02%, from about 0.015% to about 0.04%; from about 0.015% to about 0.03%, from about 0.015% to about 0.02%, from about 0.02% to about 0.04%, from about 0.02% to about 0.035%, or from about 0.02% to about 0.03%. In specific embodiments, the surfactant is present in an amount of about 0.02%. In alternative embodiments, the surfactant is present in an amount of about 0.01%, about 0.015%, about 0.025%, about 0.03%, about 0.035%, or about 0.04%. In alternative embodiments, the surfactant is present in an amount of about 0.4% to about 0.6% w/v.

In exemplary embodiments of the invention, the surfactant is a nonionic surfactant selected from the group consisting of: Polysorbate 20, Polysorbate 80 and F127. In some embodiments, the surfactant is Polysorbate 80.

In specific embodiments, the PD-1 formulations of the invention comprise about 0.01% to about 0.04% PS80. In further embodiments, the formulations of the invention comprise PS80 in an amount of about 0.008%, about 0.01%, about 0.015%, about 0.02%, about 0.025%, about 0.03%, about 0.035%, about 0.04% or about 0.045%. In particular embodiments, the formulations of the invention comprise about 0.02% PS80.

In some embodiments of the invention, the surfactant in the anti-human PD-1 antibody formulation is a poloxamer and the stabilizer is sucrose or trehalose.

In some embodiments of the invention, the surfactant in the anti-human PD-1 antibody formulation is a poloxamer and the stabilizer is sucrose or trehalose.

In some embodiments of the invention, the surfactant in the anti-human PD-1 antibody formulation is P188 and the stabilizer is sucrose or trehalose.

In some embodiments of the invention, the surfactant in the anti-human PD-1 antibody formulation is P388 and the stabilizer is sucrose or trehalose.

In some embodiments of the invention, the surfactant in the anti-human PD-1 antibody formulation is P407 and the stabilizer is sucrose or trehalose.

In some embodiments of the invention, the surfactant in the anti-human PD-1 antibody formulation is DDAO and the stabilizer is sucrose or trehalose.

The invention also provides an anti-human PD-1 antibody formulation as described herein, wherein the formulation is contained in a glass vial or injection device (e.g. a syringe).

In embodiments of the formulations of the invention, the anti-human PD-1 antibody formulation has one or more of the following attributes after storage at 2-8° C. for 10 days:
a) the % mAb as measured by reducing UP-SEC is ≥95.5%,
b) the % high molecular weight species as measured by UP-SEC<0.5%, and
c) the % monomer as measured by UP-SEC is ≥99%, ≥99.1%, ≥99.2%, ≥99.3%, ≥99.4%, or ≥99.5%.

In embodiments of the formulations of the invention, the anti-human PD-1 antibody formulation has one or more of the following attributes after storage at 50° C. for 10 days:
a) the % mAb as measured by reducing UP-SEC is ≥94%,
b) the % high molecular weight species as measured by UP-SEC≤7%, ≤6.5%, ≤6%, ≤5.5%, ≤5%, and
c) the % monomer as measured by UP-SEC is ≥94%.

In embodiments of invention, the anti-human PD-1 antibody formulation is stable for at least 10 days at 50° C.

### Specific Aspects and Embodiments of the Invention

In one aspect (A1), the invention provides an anti-human PD-1 antibody formulation, comprising: (a) about 5 mg/mL to about 250 mg/mL of an anti-human PD-1 antibody, or antigen binding fragment thereof; (b) about 5 mM to about 20 mM buffer; (c) about 1.5 to about 8.0% weight/volume (w/v) stabilizer selected from the group consisting of: a non-reducing sugar or (2-hydroxypropyl)-β-cyclodextrin, mannitol, sorbitol, L-arginine, a pharmaceutically acceptable salt of L-arginine, L-proline, a pharmaceutically acceptable salt of L-proline, L-histidine, a pharmaceutically acceptable salt of L-histidine, glycine, and a pharmaceutically acceptable salt of glycine; (d) about 0.005% w/v to about 0.60% w/v non-ionic surfactant, about 0.23% w/v to about 1.15% w/v ionic surfactant (other than DDAO); or about 0.005% w/v to about 0.20% w/v dimethyl-dodecylamine oxide (DDAO); and (e) about 1 mM to about 30 mM anti-oxidant.

In one embodiment of aspect (A1), the buffer is histidine or acetate.

In one embodiment of aspect (A1), the buffer is about 10 mM histidine.

In one embodiment of aspect (A1), the buffer is about 10 mM L-histidine.

In one embodiment of aspect (A1), the buffer is about 10 mM acetate.

In one embodiment of aspect (A1), the stabilizer is about 6% to about 8% w/v non-reducing sugar.

In one embodiment of aspect (A1), the stabilizer is about 6% to about 8% w/v sucrose.

In one embodiment of aspect (A1), the stabilizer is about 6% to about 8% w/v trehalose.

In one embodiment of aspect (A1), the stabilizer is about 6% to about 8% w/v (2-hydroxypropyl)-β-cyclodextrin.

In one embodiment of aspect (A1), the stabilizer is about 3% to about 5% w/v mannitol.

In one embodiment of aspect (A1), the stabilizer is about 3% to about 5% w/v sorbitol.

In one embodiment of aspect (A1), the stabilizer is about 3% to about 5% w/v L-arginine, or a pharmaceutically acceptable salt thereof.

In one embodiment of aspect (A1), the stabilizer is about 3% to about 5% w/v L-arginine, or a pharmaceutically acceptable salt thereof, and the pH of the formulation is from about 6.0 to about 6.5.

In one embodiment of aspect (A1), the stabilizer is about 1.9% to about 3.3% w/v L-arginine, or a pharmaceutically acceptable salt thereof.

In one embodiment of aspect (A1), the stabilizer is about 1.9% to about 2.3% w/v L-arginine, or a pharmaceutically acceptable salt thereof.

In one embodiment of aspect (A1), the stabilizer is about 2.9% to about 3.3% w/v L-arginine, or a pharmaceutically acceptable salt thereof.

In one embodiment of aspect (A1), the stabilizer is about 3% to about 5% w/v L-arginine.

In one embodiment of aspect (A1), the stabilizer is about 3% to about 5% w/v arginine-HCl.

In one embodiment of aspect (A1), the stabilizer is about 3% to about 5% w/v L-proline, or a pharmaceutically acceptable salt thereof.

In one embodiment of aspect (A1), the stabilizer is about 3% to about 5% w/v L-proline.

In one embodiment of aspect (A1), the stabilizer is about 3% to about 5% w/v L-proline HCl.

In one embodiment of aspect (A1), the stabilizer is about 1.5% to about 1.9% w/v L-proline, or a pharmaceutically acceptable salt thereof.

In one embodiment of aspect (A1), the stabilizer is about 1.5% to about 1.9% w/v L-proline.

In one embodiment of aspect (A1), the stabilizer is about 1.5% to about 1.9% w/v L-proline HCl.

In one embodiment of aspect (A1), the stabilizer is about 2% to about 3% w/v L-histidine, or a pharmaceutically acceptable salt thereof.

In one embodiment of aspect (A1), the stabilizer is about 2% to about 3% w/v L-histidine.

In one embodiment of aspect (A1), the stabilizer is about 2% to about 3% w/v L-histidine HCl.

In one embodiment of aspect (A1), the stabilizer is about 160 mM to about 200 mM glycine, or a pharmaceutically acceptable salt thereof.

In one embodiment of aspect (A1), the stabilizer is about 1.8 to about 2.2% w/v glycine, or pharmaceutically acceptable salt thereof.

In one embodiment of aspect (A1), the anti-oxidant is about 1 mM to about 20 mM L-methionine or a pharmaceutically acceptable salt thereof.

In one embodiment of aspect (A1), the anti-oxidant is about 5 mM to about 15 mM L-methionine or a pharmaceutically acceptable salt thereof.

In one embodiment of aspect (A1), the anti-oxidant is about 10 mM L-methionine or a pharmaceutically acceptable salt thereof.

In one embodiment of aspect (A1), the anti-oxidant is L-methionine.

In one embodiment of aspect (A1), the formulation comprises greater than 200 mg/mL of the anti-human PD-1 antibody, or antigen binding fragment thereof.

In one embodiment of aspect (A1), the surfactant is: (a) about 0.23% w/v to about 1.15% w/v sodium dodecyl sulfate; (b) about 0.005% w/v to about 0.60% w/v non-ionic surfactant, which is selected from the group consisting of: polysorbate 20, a poloxamer, vitamin E D-α-tocopherol polyethylene glycol succinate (TPGS), polyethylene glycol tert-octylphenyl ether, and n-octyl β-D-maltoside (OM), or (c) about 0.005% w/v to about 0.20% w/v dimethyl-dodecylamine oxide (DDAO).

In one embodiment of aspect (A1), the surfactant is: (a) about 0.23% w/v to about 1.15% w/v sodium dodecyl sulfate; (b) about 0.005% w/v to about 0.60% w/v non-ionic surfactant, which is selected from the group consisting of: poloxamer 338 (P338), poloxamer 407 (P407), vitamin E D-α-tocopherol polyethylene glycol succinate (TPGS), n-dodecyl β-D-maltoside (DDM) and n-octyl β-D-maltoside (OM), or (c) about 0.005% w/v to about 0.20% w/v dimethyl-dodecylamine oxide (DDAO).

In one embodiment of aspect (A1), the surfactant is about 0.01% to about 0.03% w/v poloxamer 338 (P338).

In one embodiment of aspect (A1), the surfactant is about 0.01% to about 0.03% w/v poloxamer 407 (P407).

In one embodiment of aspect (A1), the surfactant is about 0.01% to about 0.03% w/v vitamin E D-α-tocopherol polyethylene glycol succinate (TPGS).

In one embodiment of aspect (A1), the surfactant is about 0.01% to about 0.03% w/v n-dodecyl β-D-maltoside (DDM).

In one embodiment of aspect (A1), the surfactant is about 0.4% to about 0.6% w/v n-octyl β-D-maltoside (OM).

In one embodiment of aspect (A1), the surfactant is about 0.01% to about 0.03% w/v dimethyl-dodecylamine oxide (DDAO).

In one aspect (A2), the invention provides an anti-human PD-1 antibody formulation, comprising: (a) about 5 mg/mL to about 250 mg/mL of an anti-human PD-1 antibody, or antigen binding fragment thereof; (b) about 5 mM to about 20 mM buffer; (c) a stabilizer selected from the group consisting of: (i) about 6% to about 8% weight/volume (w/v) sucrose, trehalose or (2-hydroxypropyl)β-cyclodextrin; (ii) about 3% to about 5% w/v mannitol, sorbitol, L-proline, or a pharmaceutically acceptable salt of L-proline; and (iii) about 1.8 to about 2.2% w/v glycine, or a pharmaceutically acceptable salt thereof; (d) about 0.005% to about 0.60% non-ionic surfactant or about 0.23% w/v to about 1% w/v ionic surfactant; and (e) about 1 mM to about 20 mM L-methionine or a pharmaceutically acceptable salt thereof.

In one embodiment of aspect (A2), the formulation further comprises from about 1% to about 3% w/v viscosity reducing agent.

In one embodiment of aspect (A2), the formulation further comprises from about 1% to about 3% w/v L-arginine, or a pharmaceutically acceptable salt thereof.

In one embodiment of aspect (A2), the formulation further comprises from about 1% to about 3% w/v L-lysine, or a pharmaceutically acceptable thereof.

In one embodiment of aspect (A2), the formulation further comprises from about 1% to about 3% w/v L-histidine, or a pharmaceutically acceptable thereof.

In one embodiment of aspect (A2), the formulation further comprises from about 1% to about 3% w/v L-glutamine, or a pharmaceutically acceptable thereof.

In one embodiment of aspect (A2), the formulation further comprises a mixture of two or more viscosity reducing agents selected from L-arginine, a pharmaceutically acceptable salt of L-arginine, L-lysine, a pharmaceutically acceptable of L-lysine, L-histidine, a pharmaceutically acceptable of L-histidine, L-glutamine, or a pharmaceutically acceptable of L-glutamine.

In one aspect (A3), the invention provides an anti-human PD-1 antibody formulation comprising: (a) about 200 mg/mL to about 250 mg/mL of an anti-human PD-1 antibody, or antigen binding fragment thereof; (b) about 5 mM to about 20 mM histidine buffer; (c) a stabilizer selected from the group consisting of: (i) about 6% to about 8% weight/volume (w/v) sucrose, trehalose or (2-hydroxypropyl)-β-cyclodextrin, (ii) about 3% to about 5% w/v mannitol, sorbitol, L-arginine, a pharmaceutically acceptable salt of L-arginine, L-proline, or a pharmaceutically acceptable salt of L-proline; and (iii) about 1.8 to about 2.2% w/v glycine, or a pharmaceutically acceptable salt thereof; (d) about 0.01% to about 0.04% w/v polysorbate 80; and (e) about 1 mM to about 20 mM L-methionine, or a pharmaceutically acceptable salt thereof.

In one embodiment of aspect (A3), the formulation further comprises from about 1% to about 3% w/v L-arginine, or a pharmaceutically acceptable salt thereof.

In one embodiment of aspect (A3), the formulation further comprises from about 1.25% to about 2.5% w/v L-arginine, or a pharmaceutically acceptable salt thereof.

In one embodiment of aspect (A3), the histidine buffer is present at a concentration of about 8 mM to about 12 mM.

In one embodiment of aspect (A3), the histidine buffer is L-histidine.

In one embodiment of aspect (A3), the L-methionine or pharmaceutically acceptable salt is present at a concentration of about 5 mM to about 15 mM.

In one embodiment of aspect (A3), the polysorbate 80 is present at a weight ratio of approximately 0.02% w/v.

In one embodiment of aspect (A3), the sucrose is present at a weight ratio of approximately 7% w/v.

In one aspect (A4), the invention provides an anti-human PD-1 antibody formulation, comprising: (a) about 5, 25, 75, 100 or 200 mg/mL to about 250 mg/mL of an anti-human PD-1 antibody, or antigen binding fragment thereof; (b) about 8 mM to about 12 mM histidine buffer; (c) about 5 mM to about 10 mM L-methionine, or a pharmaceutically acceptable salt thereof; (d) about 1.8 to about 8.0% w/v stabilizer selected from the group consisting of: sucrose, trehalose or (2-hydroxypropyl)-β-cyclodextrin, mannitol, sorbitol, L-arginine, a pharmaceutically acceptable salt of L-arginine, L-proline, a pharmaceutically acceptable salt of L-proline, glycine, and a pharmaceutically acceptable salt of glycine; and (e) about 0.23% w/v to about 1% w/v sodium dodecyl sulfate.

In one aspect (A5), the invention provides an anti-human PD-1 antibody formulation, comprising: (a) about 5, 25, 75, 100 or 200 mg/mL to about 250 mg/mL of an anti-human PD-1 antibody, or antigen binding fragment thereof; (b) about 8 mM to about 12 mM histidine buffer; (c) about 5 mM to about 10 mM L-methionine, or a pharmaceutically acceptable salt thereof; (d) about 1.8 to about 8.0% w/v stabilizer selected from the group consisting of: sucrose, trehalose or (2-hydroxypropyl)-β-cyclodextrin, mannitol, sorbitol, L-arginine, a pharmaceutically acceptable salt of L-arginine, L-proline, a pharmaceutically acceptable salt of L-proline, glycine, and a pharmaceutically acceptable salt of glycine; and (e) about 0.005% w/v to about 0.60% w/v non-ionic surfactant, which is selected from the group consisting of: polysorbate 20, a poloxamer, vitamin E TPGS, polyethylene glycol tert-octylphenyl ether (TRITON™ X-100), OM, DDM, and DDAO.

In one embodiment of aspect (A5), the formulation further comprises from about 1.25% to about 2.5% w/v L-arginine, or a pharmaceutically acceptable salt thereof.

In one embodiment of aspect (A5), the formulation further comprises from about 1.25% to about 2.5% w/v L-arginine.

In one embodiment of aspect (A5), the formulation further comprises from about 1.25% to about 2.5% w/v L-arginine-HCl.

In one embodiment of aspect (A5), the formulation has a pH between 5.0 and 6.0.

In one embodiment of aspect (A5), the formulation has a pH between 5.3 and 5.8.

In one aspect (A6), the invention provides an anti-human PD-1 antibody formulation, comprising: (a) about 5, 25, 75, 100 or 200 mg/mL to about 250 mg/mL of an anti-human PD-1 antibody, or antigen binding fragment thereof; (b) about 8 mM to about 12 mM histidine buffer; (c) about 5 mM to about 10 mM L-methionine, or a pharmaceutically acceptable salt thereof; (d) about 1.8 to about 8.0% w/v stabilizer selected from the group consisting of: sucrose, trehalose or (2-hydroxypropyl)-β-cyclodextrin, mannitol, sorbitol, L-arginine, a pharmaceutically acceptable salt of L-arginine, L-proline, a pharmaceutically acceptable salt of L-proline, glycine, and a pharmaceutically acceptable salt of glycine; and (e) about 0.005% w/v to about 0.20% w/v polysorbate 20.

In one embodiment of aspect (A6), the anti-human PD-1 antibody, or antigen binding fragment thereof is present at a concentration of about 100 mg/mL to about 250 mg/mL.

In one aspect (A7), the invention provides an anti-human PD-1 antibody formulation, comprising: (a) about 5, 25, 75, 100 or 200 mg/mL to about 250 mg/mL of an anti-human PD-1 antibody, or antigen binding fragment thereof; (b) about 8 mM to about 12 mM histidine buffer; (c) about 5 mM to about 10 mM L-methionine, or a pharmaceutically acceptable salt thereof; (d) about 1.8 to about 8.0% w/v stabilizer selected from the group consisting of: sucrose, trehalose or (2-hydroxypropyl)-β-cyclodextrin, mannitol, sorbitol, L-arginine, a pharmaceutically acceptable salt of L-arginine, L-proline, a pharmaceutically acceptable salt of L-proline, glycine, and a pharmaceutically acceptable salt of glycine; and (e) about 0.005% w/v to about 0.20% w/v of a poloxamer.

In one embodiment of aspect (A7), the anti-human PD-1 antibody, or antigen binding fragment thereof is present at a concentration of about 100 mg/mL to about 250 mg/mL.

In one embodiment of aspect (A7), the L-methionine or a pharmaceutically acceptable salt thereof is L-methionine-HCl.

In one embodiment of aspect (A7), the surfactant is P188.

In one embodiment of aspect (A7), the surfactant is P388.

In one embodiment of aspect (A7), the surfactant is P407.

In one aspect (A8), the invention provides an anti-human PD-1 antibody formulation, comprising: (a) about 5, 25, 75, 100 or 200 mg/mL to about 250 mg/mL of an anti-human PD-1 antibody, or antigen binding fragment thereof; (b) about 8 mM to about 12 mM histidine buffer; (c) about 5 mM to about 10 mM L-methionine, or a pharmaceutically acceptable salt thereof; (d) about 1.8 to about 8.0% w/v stabilizer selected from the group consisting of: sucrose, trehalose or (2-hydroxypropyl)-β-cyclodextrin, mannitol, sorbitol, L-arginine, a pharmaceutically acceptable salt of L-arginine, L-proline, a pharmaceutically acceptable salt of L-proline, glycine, and a pharmaceutically acceptable salt of glycine; and (e) about 0.005% w/v to about 0.20% w/v vitamin E TPGS.

In one embodiment of aspect (A8), the anti-human PD-1 antibody, or antigen binding fragment thereof is present at a concentration of about 100 mg/mL to about 250 mg/mL.

In one embodiment of aspect (A8), the L-methionine or a pharmaceutically acceptable salt thereof is L-methionine-HCl.

In one aspect (A9), the invention provides an anti-human PD-1 antibody formulation, comprising: (a) about 5, 25, 75, 100 or 200 mg/mL to about 250 mg/mL of an anti-human PD-1 antibody, or antigen binding fragment thereof; (b) about 8 mM to about 12 mM histidine buffer; (c) about 5 mM to about 10 mM L-methionine, or a pharmaceutically acceptable salt thereof; (d) about 1.8 to about 8.0% w/v stabilizer selected from the group consisting of: sucrose, trehalose or (2-hydroxypropyl)-β-cyclodextrin, mannitol, sorbitol, L-arginine, a pharmaceutically acceptable salt of L-arginine, L-proline, a pharmaceutically acceptable salt of L-proline, glycine, and a pharmaceutically acceptable salt of glycine; and (e) about 0.005% w/v to about 0.20% w/v TRITON™ X-100.

In one embodiment of aspect (A9), the anti-human PD-1 antibody, or antigen binding fragment thereof is present at a concentration of about 100 mg/mL to about 250 mg/mL.

In one embodiment of aspect (A9), the L-methionine or a pharmaceutically acceptable salt thereof is L-methionine-HCl.

In one aspect (A10), the invention provides an anti-human PD-1 antibody formulation, comprising: (a) about 5, 25, 75, 100 or 200 mg/mL to about 250 mg/mL of an anti-human PD-1 antibody, or antigen binding fragment thereof; (b) about 8 mM to about 12 mM histidine buffer; (c) about 5 mM to about 10 mM L-methionine, or a pharmaceutically acceptable salt thereof; (d) about 1.8 to about 8.0% w/v stabilizer selected from the group consisting of: sucrose, trehalose or (2-hydroxypropyl)-β-cyclodextrin, mannitol, sorbitol, L-arginine, a pharmaceutically acceptable salt of L-arginine, L-proline, a pharmaceutically acceptable salt of L-proline, glycine, and a pharmaceutically acceptable salt of glycine; and (e) about 0.005% w/v to about 0.20% w/v OM.

In one embodiment of aspect (A10), the anti-human PD-1 antibody, or antigen binding fragment thereof is present at a concentration of about 100 mg/mL to about 250 mg/mL.

In one embodiment of aspect (A10), the L-methionine or a pharmaceutically acceptable salt thereof is L-methionine-HCl.

In one aspect (A11), the invention provides an anti-human PD-1 antibody formulation, comprising: (a) about 5, 25, 75, 100 or 200 mg/mL to about 250 mg/mL of an anti-human PD-1 antibody, or antigen binding fragment thereof; (b) about 8 mM to about 12 mM histidine buffer; (c) about 5 mM to about 10 mM L-methionine, or a pharmaceutically acceptable salt thereof; (d) about 1.8 to about 8.0% w/v stabilizer selected from the group consisting of: sucrose, trehalose or (2-hydroxypropyl)-β-cyclodextrin, mannitol, sorbitol, L-arginine, a pharmaceutically acceptable salt of L-arginine, L-proline, a pharmaceutically acceptable salt of L-proline, glycine, and a pharmaceutically acceptable salt of glycine; and (e) about 0.005% w/v to about 0.20% w/v DDM.

In one embodiment of aspect (A11), the anti-human PD-1 antibody, or antigen binding fragment thereof is present at a concentration of about 100 mg/mL to about 250 mg/mL.

In one embodiment of aspect (A11), the L-methionine or a pharmaceutically acceptable salt thereof is L-methionine-HCl.

In one aspect (A12), the invention provides an anti-human PD-1 antibody formulation, comprising: (a) about 5, 25, 75, 100 or 200 mg/mL to about 250 mg/mL of an anti-human PD-1 antibody, or antigen binding fragment thereof; (b) about 8 mM to about 12 mM histidine buffer; (c) about 5 mM to about 10 mM L-methionine, or a pharmaceutically acceptable salt thereof; (d) about 1.8 to about 8.0% w/v stabilizer selected from the group consisting of: sucrose, trehalose or (2-hydroxypropyl)-β-cyclodextrin, mannitol, sorbitol, L-arginine, a pharmaceutically acceptable salt of L-arginine, L-proline, a pharmaceutically acceptable salt of L-proline, glycine, and a pharmaceutically acceptable salt of glycine; and (e) about 0.005% w/v to about 0.20% w/v DDAO.

In one embodiment of aspect (A12), the anti-human PD-1 antibody, or antigen binding fragment thereof is present at a concentration of about 100 mg/mL to about 250 mg/mL.

In one embodiment of aspect (A12), the L-methionine or a pharmaceutically acceptable salt thereof is L-methionine-HCl.

In one aspect (A13), the invention provides an anti-human PD-1 antibody formulation, comprising: (a) about 5, 25, 75, 100 or 200 mg/mL to about 250 mg/mL of an anti-human PD-1 antibody, or antigen binding fragment thereof; (b) about 8 mM to about 12 mM histidine buffer; (c) about 5 mM to about 10 mM L-methionine, or a pharmaceutically acceptable salt thereof; (d) about 1.8 to about 8.0% w/v sucrose; and (e) about 0.005% w/v to about 0.60% w/v non-ionic surfactant, which is selected from the group consisting of: polysorbate 20, a poloxamer, vitamin E TPGS, polyethylene glycol tert-octylphenyl ether (TRITON™ X-100), OM, DDM, and DDAO.

In one embodiment of aspect (A13), the anti-human PD-1 antibody, or antigen binding fragment thereof is present at a concentration of about 75 mg/mL to about 250 mg/mL.

In one embodiment of aspect (A13), the sucrose is present in a concentration of about 6% to about 8%.

In one aspect (A14), the invention provides an anti-human PD-1 antibody formulation, comprising: (a) about 75, 100 or 200 mg/mL to about 250 mg/mL of an anti-human PD-1 antibody, or antigen binding fragment thereof; (b) about 10 mM histidine buffer; (c) about 10 mM L-methionine, or a pharmaceutically acceptable salt thereof; (d) about 7.0% w/v sucrose; and (e) about 0.005% w/v to about 0.60% w/v non-ionic surfactant, which is selected from the group consisting of: polysorbate 20, a poloxamer, vitamin E TPGS, polyethylene glycol tert-octylphenyl ether (TRITON™ X-100), OM, DDM, and DDAO.

In one embodiment of aspect (A13) or (A14), the non-ionic surfactant is polysorbate 20.

In one embodiment of aspect (A13) or (A14), the non-ionic surfactant is a polomaxer.

In one embodiment of aspect (A13) or (A14), the non-ionic surfactant is vitamin E TPGS.

In one embodiment of aspect (A13) or (A14), the non-ionic surfactant is polyethylene glycol tert-octylphenyl ether (TRITON™ X-100).

In one embodiment of aspect (A13) or (A14), the non-ionic surfactant is OM.

In one embodiment of aspect (A13) or (A14), the non-ionic surfactant is DDM.

In one embodiment of aspect (A13) or (A14), the non-ionic surfactant is DDAO.

In one embodiment of any of aspects (A1)-(A14), the formulation has a pH between 4.5 and 6.5.

In one embodiment of any of aspects (A1)-(A14), the formulation has a pH between 5.0 and 6.0.

In one embodiment of any of aspects (A1)-(A14), the formulation has a pH between 5.3 and 5.8.

In one embodiment of any of aspects (A1)-(A14), the formulation has a pH around 5.5.

In one embodiment of any of aspects (A1)-(A14), the formulation further comprises a metal chelator.

In one embodiment of any of aspects (A1)-(A14), the formulation further comprises DTPA, which is present at a concentration of about 10 μM to about 30 μM.

In some embodiments of any of aspects (A1)-(A14), the formulation is a liquid.

In some embodiments of any of aspects (A1)-(A14), the formulation is a reconstituted solution from a lyophilized formulation.

In any of the specific aspects and embodiments described herein, any anti-PD-1 antibody or antigen binding fragment thereof (i.e. an antibody or antigen binding fragment that specifically binds human PD-1, e.g. pembrolizumab or an antigen-binding fragment thereof) can be used. In particular embodiments, one of the anti-PD-1 antibodies, or antigen binding fragments thereof, described herein, e.g. described in the section entitled Anti-PD-1 Antibodies and Antigen-Binding Fragments Thereof, is used.

In some embodiments of the invention, any of the formulations described herein is in aqueous solution. In alternative embodiment, the invention provides lyophilized formulations made by lyophilizing an aqueous formulation to provide a reconstituted formulation of the invention, as discussed more fully, infra.

### Lyophilized Pharmaceutical Compositions

Lyophilized formulations of therapeutic proteins provide several advantages. Lyophilized formulations in general offer better chemical stability than solution formulations, and thus increased half-life. A lyophilized formulation may also be reconstituted at different concentrations depending on clinical factors, such as route of administration or dosing. For example, a lyophilized formulation may be reconstituted at a high concentration (i.e. in a small volume) if necessary for subcutaneous administration, or at a lower concentration if administered intravenously. High concentrations may also be necessary if high dosing is required for a particular subject, particularly if administered subcutaneously where injection volume must be minimized. One such lyophilized antibody formulation is disclosed at U.S. Pat. No. 6,267,958, which is hereby incorporated by reference in its entirety. Lyophilized formulations of another therapeutic protein are disclosed at U.S. Pat. No. 7,247,707, which is hereby incorporated by reference in its entirety.

Typically, the lyophilized formulation is prepared in anticipation of reconstitution at high concentration of drug product (DP, in an exemplary embodiment humanized anti-PD-1 antibody pembrolizumab, or antigen binding fragment thereof), i.e. in anticipation of reconstitution in a low volume of water. Subsequent dilution with water or isotonic buffer can then readily be used to dilute the DP to a lower concentration. Typically, excipients are included in a lyophilized formulation of the invention at levels that will result in a roughly isotonic formulation when reconstituted at high DP concentration, e.g. for subcutaneous administration. Reconstitution in a larger volume of water to give a lower DP concentration will necessarily reduce the tonicity of the reconstituted solution, but such reduction may be of little significance in non-subcutaneous, e.g. intravenous, administration. If isotonicity is desired at lower DP concentration, the lyophilized powder may be reconstituted in the standard low volume of water and then further diluted with isotonic diluent, such as 0.9% sodium chloride.

In one embodiment of the invention, humanized anti-PD-1 antibody (or antigen binding fragment thereof) is formulated as a lyophilized powder for reconstituting and utilizing for intravenous administration. In certain embodiments, the antibody (or antigen binding fragment thereof) is provided at about 50 mg/vial, and is reconstituted with sterile water for injection prior to use. If desired, the reconstituted antibody may be aseptically diluted with 0.9% sodium chloride Injection USP in a sterile IV container. In some embodiments, the target pH of the reconstituted formulation is 5.5±0.5. In various embodiments, the lyophilized formulation of the invention enables reconstitution of the anti-PD-1 antibody to high concentrations, such as about 20, 25, 30, 40, 50, 60, 75, 100, 125, 150, 175, 200, 225, 250 or more mg/mL.

Lyophilized formulations are by definition essentially dry, and thus the concept of concentration is not useful in describing them. Describing a lyophilized formulation in the terms of the weight of the components in a unit dose vial is more useful, but is problematic because it varies for different doses or vial sizes. In describing the lyophilized formulations of the invention, it is useful to express the amount of a component as the ratio of the weight of the component compared to the weight of the drug substance (DS) in the same sample (e.g. a vial). This ratio may be expressed as a percentage. Such ratios reflect an intrinsic property of the lyophilized formulations of the invention, independent of vial size, dosing, and reconstitution protocol.

In other embodiments, the lyophilized formulation of anti-human PD-1 antibody, or antigen binding fragment, is defined in terms of the pre-lyophilization solution used to make the lyophilized formulation, such as the pre-lyophilization solution. In one embodiment the pre-lyophilization solution comprises antibody, or antigen-binding fragment thereof, at a concentration of about 10 mg/mL about 25 mg/mL or about 50 mg/mL. Such pre-lyophilization solutions may be at pH 4.4-5.2 (including about 4.4, 4.5, 4.6, 4.7, 4.8, 4.9, 5.0, 5.1. and 5.2), e.g. preferably about pH 4.8, or about pH 5.5.

In yet other embodiments, the lyophilized formulation of anti-human PD-1 antibody, or antigen binding fragment, is defined in terms of the reconstituted solution generated from the lyophilized formulation.

Reconstituted solutions may comprise antibody, or antigen-binding fragment thereof, at concentrations of about 10, 15, 20, 25, 30, 40, 50, 60, 75, 80, 90 or 100 mg/mL or higher concentrations such as 150 mg/mL, 167 mg/mL, 200 mg/mL, or up to about 250 mg/mL. Such reconstituted solutions may be at about pH 5.5, or range from about pH 5.0 to about 6.0

The lyophilized formulations of the invention are formed by lyophilization (freeze-drying) of a pre-lyophilization solution. Freeze-drying is accomplished by freezing the formulation and subsequently subliming water at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will dictate the time required for drying, which can range from a few hours to several days (e.g. 40-60 hrs). A secondary drying stage may be carried out at about 0-40° C., depending primarily on the type and size of container and the type of protein employed. The secondary drying time is dictated by the desired residual moisture level in the product and typically takes at least about 5 hours. Typically, the moisture content of a lyophilized formulation is less than about 5%, and preferably less than about 3%. The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial.

The lyophilized formulations of the invention are reconstituted prior to administration. The protein may be reconstituted at a concentration of about 10, 15, 20, 25, 30, 40, 50, 60, 75, 80, 90 or 100 mg/mL or higher concentrations such as 150 mg/mL, 200 mg/mL, 250 mg/mL, or 300 mg/mL up to about 500 mg/mL. High protein concentrations are particularly useful where subcutaneous delivery of the reconstituted formulation is intended. However, for other routes of administration, such as intravenous administration, lower concentrations of the protein may be desired (e.g. from about 5-50 mg/mL).

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution.

The invention provides a liquid anti-human PD-1 antibody formulation that is reconstituted from a lyophilized formulation wherein the reconstituted solution comprises: a) about 125 mg/mL to about 175 mg/mL of an anti-human PD-1 antibody, or antigen binding fragment thereof; b) about 8 mM to about 12 mM histidine buffer; c) a stabilizer selected from the group consisting of: (i) about 3% to about 8% weight/volume (w/v) sucrose; (ii) about 2% to about 5% w/v L-arginine, or a pharmaceutically acceptable salt thereof (iii) about 3% to about 5% mannitol and about 1% to about 2% sucrose, and (iv) a combination of i) and ii); and d) about 0.01% to about 0.04% polysorbate 80.

In embodiments of the invention, the stabilizer comprises about 3% to about 8% weight/volume (w/v) sucrose.

In embodiments of the invention, the stabilizer comprises about 2% to about 5% w/v L-arginine, or a pharmaceutically acceptable salt thereof.

In embodiments of the invention, the stabilizer comprises about 3% to about 5% mannitol and about 1% to about 2% sucrose.

In embodiments of the invention, the stabilizer comprises about 4% to about 4.5% mannitol and about 1% to about 2% sucrose.

In embodiments of the invention, the stabilizer comprises about 3% to about 8% weight/volume (w/v) sucrose and about 2% to about 5% w/v L-arginine, or a pharmaceutically acceptable salt thereof. In specific embodiments, the stabilizer comprises sucrose and L-arginine. In other embodiments, the stabilizer comprises sucrose and L-arginine-HCl.

In specific embodiments, the stabilizer comprises a combination of 2-4% w/v L-arginine, or a pharmaceutically acceptable salt thereof and 3.5-6% w/v sucrose. In other embodiments, the stabilizer comprises a combination of about 3% L-arginine or a pharmaceutically acceptable salt thereof and about 5.5% sucrose. In other embodiments, the stabilizer comprises a combination of about 2% L-arginine or a pharmaceutically acceptable salt thereof and about 5% sucrose. In other embodiments, the stabilizer comprises a combination of about 2% L-arginine or a pharmaceutically acceptable salt thereof and about 3.7% sucrose.

### Liquid Pharmaceutical Compositions

A liquid antibody formulation can be made by taking the drug substance (e.g., anti-humanized PD-1) which is in liquid form (e.g., pembrolizumab in an aqueous pharmaceutical formulation) and buffer exchanging it into the desired buffer as the last step of the purification process. There is no lyophilization step in this embodiment. The drug substance in the final buffer is concentrated to a desired concentration. Excipients such as sucrose, methionine and polysorbate 80 are added to the drug substance and it is diluted using the appropriate buffer to final protein concentration. The final formulated drug substance is filtered, e.g. using 0.22 μm filters, and filled into a final container (e.g. glass vials or syringes). Such a liquid formulation is exemplified by a final liquid formulation comprising 10 mM histidine pH 5.5, 7% sucrose, 0.02% polysorbate 80, 25-200 mg/mL pembrolizumab and 1.5-2.5% arginine, or a pharmaceutically acceptable salt thereof.

### III. Methods of Use

The invention also relates to a method of treating cancer in a subject, the method comprising administering an effective amount of any of the formulations of the invention; i.e., any formulation described herein (including the formulations of the invention defined as aspects (A1)-(A14) in the Specific Aspects and Embodiments of the Invention section herein (referred to hereafter as “aspects (A1)-(A14)”)), to the subject. In some embodiments of this method, the formulation is administered to the subject via intravenous administration. In other embodiments, the formulation is administered to the subject by subcutaneous administration.

In any of the methods of the invention, the cancer can be selected from the group consisting of: melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, salivary cancer, prostate cancer (e.g. hormone refractory prostate adenocarcinoma), pancreatic cancer, colon cancer, esophageal cancer, liver cancer, thyroid cancer, glioblastoma, glioma, and other neoplastic malignancies.

In some embodiments the lung cancer in non-small cell lung cancer.

In alternate embodiments, the lung cancer is small-cell lung cancer.

In some embodiments, the lymphoma is Hodgkin lymphoma.

In other embodiments, the lymphoma is non-Hodgkin lymphoma. In particular embodiments, the lymphoma is mediastinal large B-cell lymphoma. In some embodiments, the lymphoma is diffuse large B-cell lymphoma (DLBCL).

In some embodiments, the breast cancer is triple negative breast cancer.

In further embodiments, the breast cancer is ER+/HER2− breast cancer.

In some embodiments, the bladder cancer is urothelial cancer.

In some embodiments, the head and neck cancer is nasopharyngeal cancer. In some embodiments, the cancer is thyroid cancer. In other embodiments, the cancer is salivary cancer. In other embodiments, the cancer is squamous cell carcinoma of the head and neck.

In some embodiments, the cancer is metastatic colorectal cancer with high levels of microsatellite instability (MSI-H).

In some embodiments, the cancer is a solid tumor with a high level of microsatellite instability (MSI-H).

In some embodiments, the cancer is a solid tumor with a high mutational burden.

In some embodiments, the cancer is selected from the group consisting of: melanoma, non-small cell lung cancer, relapsed or refractory classical Hodgkin lymphoma, head and neck squamous cell carcinoma, urothelial cancer, esophageal cancer, gastric cancer, DLBCL and hepatocellular cancer.

In other embodiments of the above treatment methods, the cancer is a Heme malignancy. In certain embodiments, the Heme malignancy is acute lymphoblastic leukemia (ALL), acute myeloid leukemia (AML), chronic lymphocytic leukemia (CLL), chronic myeloid leukemia (CIVIL), DLBCL, EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin lymphoma (NHL), or small lymphocytic lymphoma (SLL).

Malignancies that demonstrate improved disease-free and overall survival in relation to the presence of tumor-infiltrating lymphocytes in biopsy or surgical material, e.g. melanoma, colorectal, liver, kidney, stomach/esophageal, breast, pancreas, and ovarian cancer are encompassed in the methods and treatments described herein. Such cancer subtypes are known to be susceptible to immune control by T lymphocytes. Additionally, included are refractory or recurrent malignancies whose growth may be inhibited using the antibodies described herein.

In some embodiments, the formulations of the invention (e.g. aspects (A1)-(A14)) are administered to a subject having a cancer characterized by elevated expression of PD-L1 and/or PD-L2 in tested tissue samples, including: ovarian, renal, colorectal, pancreatic, breast, liver, gastric, esophageal cancers and melanoma. Additional cancers that can benefit from treatment with anti-PD-1 antibodies such as humanized anti-PD-1 antibody pembrolizumab include those associated with persistent infection with viruses such as human immunodeficiency viruses, hepatitis viruses class A, B and C, Epstein Barr virus, human papilloma viruses that are known to be causally related to for instance Kaposi's sarcoma, liver cancer, nasopharyngeal cancer, lymphoma, cervical, vulval, anal, penile and oral cancers.

In one embodiment, the invention comprises a method of treating cancer in a human patient comprising administering any formulation of the invention (e.g. aspects (A1)-(A14)) to the patient.

In one embodiment, the invention comprises a method of treating unresectable or metastatic melanoma in a human patient comprising administering any formulation of the invention (e.g. aspects (A1)-(A14)) to the patient.

In one embodiment, the invention comprises a method of treating metastatic non-small cell lung cancer (NSCLC) in a human patient comprising administering a formulation of the invention (e.g. aspects (A1)-(A14)) to the patient. In specific embodiments, the patient has a tumor with high PD-L1 expression [(Tumor Proportion Score (TPS)≥50%)] and was not previously treated with platinum-containing chemotherapy. In other embodiments, the patient has a tumor with PD-L1 expression (TPS≥1%) and was previously treated with platinum-containing chemotherapy. In still other embodiments, the patient has a tumor with PD-L1 expression (TPS≥1%) and was not previously treated with platinum-containing chemotherapy. In specific embodiments, the patient had disease progression on or after receiving platinum-containing chemotherapy.

In certain embodiments, the PD-L1 TPS is determined by an FDA-approved test.

In certain embodiments, the patient's tumor has no EGFR or ALK genomic aberrations.

In certain embodiments, the patient's tumor has an EGFR or ALK genomic aberration and had disease progression on or after receiving treatment for the EGFR or ALK aberration(s) prior to receiving the anti-PD-1 antibody, or antigen binding fragment thereof.

In one embodiment, the invention comprises a method of treating metastatic non-small cell lung cancer (NSCLC) in a human patient comprising: (1) administering a formulation of the invention (e.g. aspects (A1)-(A14)) to the patient, and (2) administering pemetrexed and carboplatin to the patient. In specific embodiments, the patient was not previously treated with an anti-cancer therapeutic prior to starting the combination treatment regimen with the formulation of the invention, pemetrexed and carboplatin.

In certain embodiments, the patient has non-squamous non-small cell lung cancer.

In certain embodiments, pemetrexed is administered to the patient in an amount of 500 mg/m². In sub-embodiments, pemetrexed is administered to the patient via intravenous infusion every 21 days. In specific embodiments, the infusion time is about 10 minutes.

In embodiments of the invention where the patient is treated with a formulation of the invention in combination with pemetrexed, the invention further comprises administering about 400 μg to about 1000 μg of folic acid to the patient once per day, beginning about 7 days prior to administering pemetrexed to the patient and continuing until about 21 days after the patient is administered the last dose of pemetrexed. In certain embodiments the folic acid is administered orally. In some embodiments, the invention further comprises administering about 1 mg of vitamin B12 to the patient about 1 week prior to the first administration of pemetrexed and about every three cycles of pemetrexed administration (i.e., approximately every 9 weeks). In certain embodiments the vitamin B12 is administered intramuscularly. In certain embodiments, the invention further comprises administering about 4 mg of dexamethasone to the patient twice a day on the day before, the day of, and the day after pemetrexed administration. In certain embodiments the dexamethasone is administered orally.

In certain embodiments, the patient has squamous non-small cell lung cancer.

In one embodiment, the invention comprises a method of treating metastatic squamous NSCLC in a human patient comprising: (1) administering a formulation of the invention (e.g. aspects (A1)-(A14)) to the patient, (2) administering carboplatin and (3) administering paclitaxel or nab-paclitaxel to the patient.

In one embodiment, the invention comprises a method of treating recurrent or metastatic head and neck squamous cell cancer (HNSCC) in a human patient comprising administering any formulation of the invention (e.g. aspects (A1)-(A14)) to the patient. In certain embodiments, the patient was previously treated with platinum-containing chemotherapy. In certain embodiments, the patient had disease progression on or after platinum-containing chemotherapy.

In one embodiment, the invention comprises a method of treating Merkel cell carcinoma in a human patient comprising administering a formulation of the invention (e.g. aspects (A1)-(A14)) to the patient.

In one embodiment, the invention comprises a method of treating refractory classical Hodgkin lymphoma (cHL) in a human patient comprising administering a formulation of the invention (e.g. aspects (A1)-(A14)) to the patient. In certain embodiments, the patient has relapsed after 3 or more lines of therapy for cHL. In specific embodiments, the patient is an adult patient. In alternative embodiments, the patient is a pediatric patient.

In one embodiment, the invention comprises a method of treating locally advanced or metastatic urothelial carcinoma in a human patient comprising administering a formulation of the invention (e.g. aspects (A1)-(A14)) to the patient. In certain embodiments, the patient is not eligible for cisplatin-containing chemotherapy. In certain embodiments, the patient has disease progression during or following platinum-containing chemotherapy or within 12 months of neoadjuvant or adjuvant treatment with platinum-containing chemotherapy.

In one embodiment, the invention comprises a method of treating high-risk non-muscle invasive bladder cancer in a human patient comprising administering a formulation of the invention (e.g. aspects (A1)-(A14)) to the patient. In one embodiment, the patient has carcinoma in situ (CIS) or CIS with papillary disease. In one embodiment, the patient was previously treated with a standard therapy.

In one embodiment, the invention comprises a method of treating hepatocellular carcinoma (HCC) in a human patient comprising administering a formulation of the invention (e.g. aspects (A1)-(A14)) to the patient. In one embodiment, the patient has advanced HCC. In one embodiment, the patient was previously treated with sorafenib. In one embodiment, the patient has disease progression or intolerance of sorafenib. In one embodiment, the patient had not received treatment for systemic disease prior to treatment with a formulation of the invention.

In one embodiment, the invention comprises a method of treating unresectable or metastatic, microsatellite instability-high (MSI-H) or mismatch repair deficient solid tumors in a human patient comprising administering a formulation of the invention (e.g. aspects (A1)-(A14)) to the patient. In specific embodiments, the patient had disease progression following prior anti-cancer treatment.

In one embodiment, the invention comprises a method of treating unresectable or metastatic, microsatellite instability-high (MSI-H) or mismatch repair deficient colorectal cancer in a human patient comprising administering a formulation of the invention (e.g. aspects (A1)-(A14)) to the patient. In specific embodiments, the patient had disease progression following prior treatment with a fluoropyrimidine, oxaliplatin, and irinotecan.

In one embodiment, the invention comprises a method of treating recurrent locally advanced or metastatic gastric cancer in a human patient comprising administering a formulation of the invention (e.g. aspects (A1)-(A14)) to the patient.

In one embodiment, the invention comprises a method of treating recurrent locally advanced or metastatic gastroesophageal junction adenocarcinoma in a human patient comprising administering a formulation of the invention (e.g. aspects (A1)-(A14)) to the patient. In specific embodiments, the patient's tumor expresses PD-L1 [Combined Positive Score (CPS)≥1]. In specific embodiments, the patient has disease progression on or after two or more prior lines of therapy including fluoropyrimidine- and platinum-containing chemotherapy. In specific embodiments, the patient has disease progression on or after two or more prior lines of therapy including HER2/neu-targeted therapy.

In one embodiment, the invention comprises a method of treating cancer in a human patient comprising administering a formulation of the invention (e.g. aspects (A1)-(A14)) to the patient, wherein the patient has a cancer selected from the group consisting of: melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, and salivary cancer.

In one embodiment, the invention comprises a method of treating small cell lung cancer in a human patient comprising administering a formulation of the invention (e.g. aspects (A1)-(A14)) to the patient.

In one embodiment, the invention comprises a method of treating esophageal cancer in a human patient comprising administering a formulation of the invention (e.g. aspects (A1)-(A14)) to the patient. In one embodiment, the patient was previously treated with one, two, or three standard therapies. In one embodiment, the patient has advanced or metastatic adenocarcinoma or squamous cell carcinoma of the esophagus. In one embodiment, the patient has advanced or metastatic Siewert type I adenocarcinoma of the esophagogastric junction.

In one embodiment, the invention comprises a method of treating non-Hodgkin lymphoma in a human patient comprising administering a formulation of the invention (e.g. aspects (A1)-(A14)) to the patient. In specific embodiments, the non-Hodgkin lymphoma is mediastinal large B-cell lymphoma. In specific embodiments, the non-Hodgkin lymphoma is diffuse large B-cell lymphoma.

In one embodiment, the invention comprises a method of treating breast cancer in a human patient comprising administering a formulation of the invention (e.g. aspects (A1)-(A14)) to the patient. In certain embodiments, the breast cancer is triple negative breast cancer. In certain embodiments, the breast cancer is ER+/HER2−|breast cancer.

In one embodiment, the invention comprises a method of treating nasopharyngeal cancer in a human patient comprising administering a formulation of the invention (e.g. aspects (A1)-(A14)) to the patient.

In one embodiment, the invention comprises a method of treating thyroid cancer in a human patient comprising administering a formulation of the invention (e.g. aspects (A1)-(A14)) to the patient.

In one embodiment, the invention comprises a method of treating salivary cancer in a human patient comprising administering a formulation of the invention (e.g. aspects (A1)-(A14)) to the patient.

Antagonist anti-PD-1 antibodies or antibody fragments can also be used to prevent or treat infections and infectious disease. Thus, the invention provides a method for treating chronic infection in a mammalian subject comprising administering an effective amount of a formulation of the invention to the subject. In some specific embodiments of this method, the formulation is administered to the subject via intravenous administration. In other embodiments, the formulation is administered to the subject by subcutaneous administration.

These agents can be used alone, or in combination with vaccines, to stimulate the immune response to pathogens, toxins, and self-antigens. The antibodies or antigen-binding fragment thereof can be used to stimulate immune response to viruses infectious to humans, including but not limited to: human immunodeficiency viruses, hepatitis viruses class A, B and C, Epstein Barr virus, human cytomegalovirus, human papilloma viruses, and herpes viruses. Antagonist anti-PD-1 antibodies or antibody fragments can be used to stimulate immune response to infection with bacterial or fungal parasites, and other pathogens. Viral infections with hepatitis B and C and HIV are among those considered to be chronic viral infections.

The formulations of the invention may be administered to a patient in combination with one or more “additional therapeutic agents”. The additional therapeutic agent may be a biotherapeutic agent (including but not limited to antibodies to VEGF, EGFR, Her2/neu, VEGF receptors, other growth factor receptors, CD20, CD40, CD-40L, OX-40, 4-1BB, and ICOS), a growth inhibitory agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

As noted above, in some embodiments of the methods of the invention, the method further comprises administering an additional therapeutic agent. In particular embodiments, the additional therapeutic agent is an anti-LAG3 antibody or antigen binding fragment thereof, an anti-CTLA4 antibody, or antigen binding fragment thereof, an anti-GITR antibody, or antigen binding fragment thereof, an anti-TIGIT antibody, or antigen binding fragment thereof, an anti-CD27 antibody or antigen binding fragment thereof. In one embodiment, the additional therapeutic agent is a Newcastle disease viral vector expressing IL-12. In a further embodiment, the additional therapeutic agent is dinaciclib. In a further embodiment, the additional therapeutic agent is olaparib. In a further embodiment, the additional therapeutic agent is a tyrosine kinase inhibitor. In still further embodiments, the additional therapeutic agent is a STING agonist.

Suitable routes of administration may, for example, include parenteral delivery, including intramuscular, subcutaneous, as well as intrathecal, direct intraventricular, intravenous, intraperitoneal. Drugs can be administered in a variety of conventional ways, such as intraperitoneal, parenteral, intraarterial or intravenous injection. Modes of administration in which the volume of solution must be limited (e.g. subcutaneous administration) require a lyophilized formulation to enable reconstitution at high concentration.

Selecting a dosage of the additional therapeutic agent depends on several factors, including the serum or tissue turnover rate of the entity, the level of symptoms, the immunogenicity of the entity, and the accessibility of the target cells, tissue or organ in the individual being treated. The dosage of the additional therapeutic agent should be an amount that provides an acceptable level of side effects. Accordingly, the dose amount and dosing frequency of each additional therapeutic agent (e.g. biotherapeutic or chemotherapeutic agent) will depend in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Various literature references are available to facilitate selection of pharmaceutically acceptable carriers or excipients for the additional therapeutic agent. See, e.g., Remington's Pharmaceutical Sciences and U.S. Pharmacopeia: National Formulary, Mack Publishing Company, Easton, Pa. (1984); Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.

A pharmaceutical antibody formulation can be administered by continuous infusion, or by doses at intervals of, e.g., one day, 1-7 times per week, one week, two weeks, three weeks, monthly, bimonthly, etc. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

In certain embodiments, dosing will comprise administering to a subject escalating doses of 1.0, 2.0, 3.0, and 10 mg/kg of the pharmaceutical formulation, i.e, a formulation comprising pembrolizumab, over the course of treatment. The formulation comprising pembrolizumab can be a reconstituted liquid formulation, or it can be a liquid formulation not previously lyophilized. Time courses can vary, and can continue as long as desired effects are obtained. In certain embodiments, dose escalation will continue up to a dose of about 10 mg/kg. In certain embodiments, the subject will have a histological or cytological diagnosis of melanoma, or other form of solid tumor, and in certain instances, a subject may have non-measurable disease. In certain embodiments, the subject will have been treated with other chemotherapeutics, while in other embodiments, the subject will be treatment naïve.

In yet additional embodiments, the dosing regimen comprises administering a dose of 1, 2, 3, or 10 mg/kg of any of the pharmaceutical formulations described herein (i.e, a formulation of the invention), throughout the course of treatment. In certain embodiments, the interval between doses is about 21 days (±2 days).

In yet additional embodiments, the dosing regimen comprises administering a flat dose of 200 mg of any of the pharmaceutical formulations described herein (i.e, a formulation of the invention), for an interval of about 21 days (days (±2 days) throughout the course of treatment. In other embodiments, the dosing regimen comprises administering a flat dose of 400 mg of any of the pharmaceutical formulations described herein (i.e, a formulation of the invention), for an interval of about 6 weeks (±4 days) throughout the course of treatment. For such a constant dosing regimen, the interval between doses will be about 14 days (±2 days). In certain embodiments, the interval between doses will be about 21 days (±2 days).

In certain embodiments, the patient is administered an IV infusion of any of the pharmaceutical formulations described herein.

In some embodiments, the patient is administered a formulation of the invention via subcutaneous injection

In certain embodiments, the use of cell surface markers and/or cytokine markers, as described in WO2012/018538 or WO2008/156712 will be used in bioassays for monitoring, diagnostic, patient selection, and/or treatment regimens involving blockade of the PD-1 pathway. Subcutaneous administration may performed by injected using a syringe, or using other injection devices (e.g. the Injectease® device); injector pens; or needleless devices (e.g. MediJector and BioJector®).

Embodiments of the invention also include one or more of the biological formulations described herein (i) for use in, (ii) for use as a medicament or composition for, or (iii) for use in the preparation of a medicament for: (a) therapy (e.g., of the human body); (b) medicine; (c) induction of or increasing of an antitumor immune response (d) decreasing the number of one or more tumor markers in a patient; (e) halting or delaying the growth of a tumor or a blood cancer; (f) halting or delaying the progression of PD-1-related disease; (g) halting or delaying the progression cancer; (h) stabilization of PD-1-related disease; (i) inhibiting the growth or survival of tumor cells; (j) eliminating or reducing the size of one or more cancerous lesions or tumors; (k) reduction of the progression, onset or severity of PD-1-related disease; (l) reducing the severity or duration of the clinical symptoms of PD-1-related disease such as cancer (m) prolonging the survival of a patient relative to the expected survival in a similar untreated patient n) inducing complete or partial remission of a cancerous condition or other PD-1 related disease, or o) treatment of cancer.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.). Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J Biochem. 133:17-21; von Heijne (1986) Nucleic Acids Res. 14:4683-4690).

### Analytical Methods

Analytical methods suitable for evaluating the product stability include size exclusion chromatography (SEC), dynamic light scattering test (DLS), differential scanning calorimetery (DSC), iso-asp quantification, potency, UV at 340 nm, UV spectroscopy, and FTIR. SEC (J. Pharm. Sci., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Sci., 85:1290 (1996); J. Pharm. Sci., 87:1069 (1998)) measures IR spectrum in the amide one region, and gives information of protein secondary structure.

The iso-asp content in the samples is measured using the Isoquant Isoaspartate Detection System (Promega). The kit uses the enzyme protein isoaspartyl methyltransferase (PIMT) to specifically detect the presence of isoaspartic acid residues in a target protein. PIMT catalyzes the transfer of a methyl group from S-adenosyl-L-methionine to isoaspartic acid at the .alpha.-carboxyl position, generating S-adenosyl-L-homocysteine (SAH) in the process. This is a relatively small molecule, and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

All publications mentioned herein are incorporated by reference for the purpose of describing and disclosing methodologies and materials that might be used in connection with the invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### Example 1

### Materials and Methods

HP-IEX: High performance ion-exchange chromatography (HP-IEX) was used to assess the charge profile. An ion exchange HPLC method was performed using a Dionex Dionex ProPac WCX-10 column and a UV detector at 280 nm. Samples were diluted in purified water, and 80 μg were injected for analysis. The mobile phase used for the IEX analysis was a gradient of the following mobile phases (mobile phase A: 24 mM MES, pH 6, 4% acetonitrile (v/v); mobile phase B: 20 mM phosphate, 95 mM NaCl, pH 8, 4% acetonitrile (v/v). The main peak is the major component of the chromatogram and it serves as a control for the characterization of acidic and basic variants. Acidic variants elute earlier than main peak and the main cause of the formation of acidic variants is due to the deamidation of the Asn in main peak and the presence of sialic acid compared to main peak. Basic variants elute later than main peak and the main cause of the formation of basic variants is due to the incomplete removal of C-terminal Lys from the main peak. Other causes are incomplete cyclization of the N-terminal glutamine (Gln) to pyroGlu of the light chain or heavy chain or both and also due to the Isomerization of Asp in the main peak to isoAsp.

HP-SEC: Purity of the sample was assessed by size exclusion chromatography (SEC) in which the percentage of monomer was determined, as well as the percentages of high molecular weight species (HMW) and late eluting peaks (LMW species). The presence of HMW species indicates protein aggregates and the presence of LMW species indicate protein fragments. High Performance—Size Exclusion Chromatography (HP-SEC) was performed by diluting the samples to 1.0 mg/mL with water. The diluted samples were injected (10 μL) into a HPLC equipped with a YMC-pack-Diol 200 column and a UV detector. Proteins in the sample were separated by size and detected by UV absorption at 280 nm.

HP-SEC Arg: Purity of the sample was assessed by size exclusion chromatography (SEC) in which the percentage of monomer was determined, as well as the percentages of high molecular weight species (HMW) and late eluting peaks (LMW species). High Performance—Size Exclusion Chromatography (HP-SEC) was performed by diluting the samples to 5.0 mg/mL in mobile phase (50 mM sodium phosphate, 450 mM arginine mono hydrochloride, pH 7.0). The column temperature was set at 25° C. and the flow rate was maintained at 0.5 mL/min using an isocratic elution. The diluted samples were injected (30 μL) into a HPLC equipped with YMC-PACK Diol-200 column and a UV detector. Proteins in the sample were separated by size and detected by UV absorption at 280 nm.

A350: UV absorption at 350 nm was measured using 96 well plate Spectramax reader as an indication of turbidity. The absorption readings were blanked against empty plate reading and normalized for sample pathlength.

HP-HIC: High performance hydrophobic interaction chromatography (HP-HIC) was used to assess oxidized products from the non-oxidized molecule. The percentage of pre-peaks, determined to be oxidized species comprising heavy chain Met105 oxidation on one heavy chain by previous analytical characterization, as well as the percentage of the main and percentage of the post peaks were determined. A HP-HIC method was performed by diluting the sample to 5.0 mg/mL in purified water. The sample was then injected (10 μL) into an HPLC equipped with a Tosoh Phenyl-5PW column and a UV detector at 280 nm. For the HIC analysis a mobile phase containing a gradient of the following components (mobile phase A: 5 mM sodium phosphate in 2% acetonitrile, pH 7.0; mobile phase B: 400 mM ammonium sulfate, 5 mM sodium phosphate in 2% acetonitrile, pH 6.9;) was used.

VP-DSC: Valerian-Plotnikov differential scanning calorimetry (VP-DSC) can be used to determine the thermal and conformational stability of monoclonal antibodies. DSC determines the heat capacity (Cᵣ) of the protein solution relative to that of the placebo solution for increasing temperatures, producing a thermal transition upon protein unfolding. For monoclonal antibodies, multiple unfolding transitions (Tonset, Tm1, Tm2) are typically seen in the DSC thermogram corresponding to unfolding of individual domains

Dynamic Light Scattering (DLS): DLS measures the time-dependent fluctuation in light scattering intensity, which is caused by the Brownian motion of the particles. The Brownian motion is related to the size of a particle; the larger the particle, the slower the Brownian motion will be and the longer the correlation time of the time-dependent scattering fluctuations. From the scattering fluctuations, the particle size distribution profile of a sample can be derived using the Stokes-Einstein equation. From the DLS data, the hydrodynamic diameter by intensity and volume can be determined as well as the poly dispersity index (PdI) which is indicative for the presence of mixtures or higher order structures.

### Examples 2-4

Examples (2-4) highlight the preparation of formulations that use a smaller amount of antibody and excipients than intended in the final formulations. However, these formulations preserve the molar ratio of the pembrolizumab/stabilizer and pembrolizumab/surfactant of the final intended formulations. The anti-oxidant and buffer concentrations were tested at the final intended concentration. The pembrolizumab (5 mg/mL), PS80 (0.004%) and stabilizer (e.g., 1.4% sucrose) concentrations were scaled down by a factor of 5 and studied as surrogate formulations for the more concentrated, final intended formulations.

For example, formulation 1A in Table 4 has the same molar ratios as a formulation comprising 25 mg/mL pembrolizumab, 7% sucrose and 0.02% PS80. It is expected that the formulations disclosed in Examples 2-4 are representative of the intended final formulations and that the disclosed results suggest how the final higher concentration formulations would behave. It is understood that some parameters being studied, e.g. aggregation, may be impacted by the decrease in concentration due to a decreased likelihood of intermolecular interactions; however, to compensate, a more aggressive stability regimen was pursued (50° C. for 10 days) to induce and identify stability risk.

The concentration of anti-oxidant (e.g. methionine) and buffer (e.g. histidine) were not scaled down in a similar manner to the other excipients. Methionine is a functional excipient that is used to reduce the Met-105 oxidation by expunging oxidants (such as dissolved oxygen), thus maintaining the chemical stability of pembrolizumab. Since it is more challenging to maintain chemical stability at lower pembrolizumab concentrations, the methionine concentration was held constant at 10 mM. It is expected that if a specific amount of methionine is effective in the low concentration formulations, it would most likely be as effective at preventing oxidation in the higher concentration formulations. L-Histidine and/or L-Histidine hydrochloride at 10 mM is expected to maintain buffering capacity at the intended pH of the formulations tested.

Throughout Examples 2-4, the glycine used was glycine and the proline used was L-proline. (2-Hydroxypropyl)β-cyclodextrin (sold under the name CAVITRON™) is denoted as HPβC. Polysorbate 20 is denoted as PS20. Poloxamers 188, 338 and 407 (sold under the name Kolliphor® P188, P338 and 407) are denoted as P188, P338, and P407, respectively. N-dodecyl β-D-maltoside (or Lauryl-β-D-maltoside) is denoted as DDM. N-octyl β-D-maltoside (or n-octyl β-D-maltopyranoside) is denoted as OM. 4-(1,1,3,3-tetramethylbutyl)phenyl-polyethylene glycol or polyethylene glycol tert-octylphenyl ether (sold under the name TRITON™ X-100) is denoted as X100. N,N-dimethyldodecylamine N-oxide or Lauryldimethylamine N-oxide is denoted as DDAO. Sodium dodecyl sulfate or sodium lauryl sulfate is denoted as SLS.

### Example 2

Evaluation of the Stability of Low Concentration Pembrolizumab Formulations with Non-Ionic Stabilizing Surfactants in Combination with Methionine

An initial formulation study was performed to evaluate the stability of formulations comprising a low (5 mg/ml) concentration of pembrolizumab and to evaluate the impact of different formulation excipients. Stabilizer stock solutions (50 mg/mL) comprised of sucrose, trehalose, mannitol, HPβC, glycine and proline and a stock solution of methionine (20 mg/mL) were all prepared in 10 mM histidine buffer (pH 5.5). Surfactant stock solutions (˜0.32 mg/mL) comprising PS80, PS20, P188, P338, VitE TPGS, P407 (1 mg/mL) and X-100 (5 mg/mL) were prepared in 10 mM histidine buffer (pH 5.5). Protein/surfactant stock solutions of pembrolizumab and PS80, PS20, P188, P338, and VitE TPGS were prepared by combining pembrolizumab drug substance (205 mg/mL in 10 mM histidine, pH 5.5) with 10 mL of the surfactant stock solutions then QS to final volume (20 mL) with buffer to yield a protein (20 mg/mL)/surfactant (0.16 mg/mL) stock solution in 10 mM histidine buffer (pH 5.5). All stock solutions used for formulations were filtered through Millipore Express® PLUS Stericup® 0.22 μm PES filters prior to use.

Test formulations (Table 4) comprising a low concentration of pembrolizumab were prepared in a 96-well plate at a volume of 1 mL. Pembrolizumab formulations were prepared to 5 mg/mL target concentration by spiking the protein/surfactant stock solutions (for PS20, P188, P338, and VitE TPGS) with stabilizer and L-methionine stock solutions to achieve target levels and brought to final volume using histidine buffer (pH 5.5). Formulations with P407 and X100 were prepared by spiking protein, surfactant, stabilizer, and methionine stock solutions to achieve target levels of protein (5 mg/mL) and each excipient and brought to final volume using histidine buffer (pH 5.5). The well plate was covered with a 96-well silicone sealing mat and then was vacuum sealed (2×) in moisture barrier bags to minimize potential evaporation. Samples were staged in 2-8° C. (as used herein and throughout the Examples, the term “5° C.” is used interchangeably with “2-8° C.”, which indicates 5° C.±3° C. (standard deviation)) and 50° C. environmental stability chambers.

TABLE 4
Low Concentration Pembrolizumab Formulations with Non-ionic Surfactants.

| | | | Anti- | | | | Anti- |
|---|---|---|---|---|---|---|---|
| Form.¹ | Stabilizer | Surfactant | Oxidant | Form.¹ | Stabilizer | Surfactant | Oxidant |
| 1A | 1.4% sucrose | PS20 | — | 4A | 1.4% HPβC | PS20 | — |
| 1B | 1.4% sucrose | PS20 | 10 mM | 4B | 1.4% HPβC | PS20 | 10 mM |
| | | | Met | | | | Met |
| 1C | 1.4% sucrose | P188 | — | 4C | 1.4% HPβC | P188 | — |
| 1D | 1.4% sucrose | P188 | 10 mM | 4D | 1.4% HPβC | P188 | 10 mM |
| | | | Met | | | | Met |
| 1E | 1.4% sucrose | P338 | — | 4E | 1.4% HPβC | P338 | — |
| 1F | 1.4% sucrose | P338 | 10 mM | 4F | 1.4% HPβC | P338 | 10 mM |
| | | | Met | | | | Met |
| 1G | 1.4% sucrose | P407 | — | 4G | 1.4% HPβC | P407 | — |
| 1H | 1.4% sucrose | P407 | 10 mM | 4H | 1.4% HPβC | P407 | 10 mM |
| | | | Met | | | | Met |
| 1J | 1.4% sucrose | TPGS | — | 4J | 1.4% HPβC | TPGS | — |
| 1K | 1.4% sucrose | TPGS | 10 mM | 4K | 1.4% HPβC | TPGS | 10 mM |
| | | | Met | | | | Met |
| 1M | 1.4% sucrose | X100² | — | 4M | 1.4% HPβC | X100 | — |
| 1N | 1.4% sucrose | X100 | 10 mM | 4N | 1.4% HPβC | X100 | 10 mM |
| | | | Met | | | | Met |
| 2A | 0.8% mannitol | PS20 | — | 5A | 0.8% proline | PS20 | — |
| 2B | 0.8% mannitol | PS20 | 10 mM | 5B | 0.8% proline | PS20 | 10 mM |
| | | | Met | | | | Met |
| 2C | 0.8% mannitol | P188 | — | 5C | 0.8% proline | P188 | — |
| 2D | 0.8% mannitol | P188 | 10 mM | 5D | 0.8% proline | P188 | 10 mM |
| | | | Met | | | | Met |
| 2E | 0.8% mannitol | P338 | — | 5E | 0.8% proline | P338 | — |
| 2F | 0.8% mannitol | P338 | 10 mM | 5F | 0.8% proline | P338 | 10 mM |
| | | | Met | | | | Met |
| 2G | 0.8% mannitol | P407 | — | 5G | 0.8% proline | P407 | — |
| 2H | 0.8% mannitol | P407 | 10 mM | 5H | 0.8% proline | P407 | 10 mM |
| | | | Met | | | | Met |
| 2J | 0.8% mannitol | TPGS | — | 5J | 0.8% proline | TPGS | — |
| 2K | 0.8% mannitol | TPGS | 10 mM | 5K | 0.8% proline | TPGS | 10 mM |
| | | | Met | | | | Met |
| 2M | 0.8% mannitol | X100 | — | 5M | 0.8% proline | X100 | — |
| 2N | 0.8% mannitol | X100 | 10 mM | 5N | 0.8% proline | X100 | 10 mM |
| | | | Met | | | | Met |
| 3A | 1.4% trehalose | PS20 | — | 6A | 0.4% glycine | PS20 | — |
| 3B | 1.4% trehalose | PS20 | 10 mM | 6B | 0.4% glycine | PS20 | 10 mM |
| | | | Met | | | | Met |
| 3C | 1.4% trehalose | P188 | — | 6C | 0.4% glycine | P188 | — |
| 3D | 1.4% trehalose | P188 | 10 mM | 6D | 0.4% glycine | P188 | 10 mM |
| | | | Met | | | | Met |
| 3E | 1.4% trehalose | P338 | — | 6E | 0.4% glycine | P338 | — |
| 3F | 1.4% trehalose | P338 | 10 mM | 6F | 0.4% glycine | P338 | 10 mM |
| | | | Met | | | | Met |
| 3G | 1.4% trehalose | P407 | — | 6G | 0.4% glycine | P407 | — |
| 3H | 1.4% trehalose | P407 | 10 mM | 6H | 0.4% glycine | P407 | 10 mM |
| | | | Met | | | | Met |
| 3J | 1.4% trehalose | TPGS | — | 6J | 0.4% glycine | TPGS | — |
| 3K | 1.4% trehalose | TPGS | 10 mM | 6K | 0.4% glycine | TPGS | 10 mM |
| | | | Met | | | | Met |
| 3M | 1.4% trehalose | X100 | — | 6M | 0.4% glycine | X100 | — |
| 3N | 1.4% trehalose | X100 | 10 mM | 6N | 0.4% glycine | X100 | 10 mM |
| | | | Met | | | | Met |

¹All formulations comprised 5 mg/mL pembrolizumab, 10 mM histidine buffer and 0.004% surfactant, in addition to the excipients listed.
²0.04% TRITON™ X-100.

Each of the test formulations in Table 5 were visually inspected for changes in coloration or precipitate formation (data not shown). Additionally, stability of the formulations was evaluated using concentration (A280), turbidity (A350), dynamic light scattering (DLS), UP-SEC (to assess purity) and HP-IEX (charge profile) after the 10-day stability period. All formulations were considered stable at the 5° C. storage condition as supported by the UP-SEC results (Table 2) which demonstrated that there were no changes for any of the formulations that were stored at 5° C. during the 10-day time period. At 50° C., more pronounced changes were observed via UP-SEC (a decrease in % mAb) and HP-IEX (a significant decrease in % main and an increase in % acidic variants, data not shown) for each of the formulations over the time period tested. DLS data (not shown) further supported the observed decrease in % mAb (with subsequent increase in % HMW species) where a small increase in radius (nm) and % polydispersity (% Pd) were observed after the stability time period. Turbidity (A350) results for all formulations also corroborated the UP-SEC results indicating a decrease in stability of all formulations after 10 days at 50° C.

As shown in Table 5, UP-SEC measurements of formulations 1C-1F (sucrose with P188 & P338) and 3C-3F (trehalose with P188 & P338) demonstrated stability that was slightly better than the other surfactants tested over 10 days 50° C. These formulations showed smaller % changes in monomer (UP-SEC) suggesting increased stability over other combinations. There were no appreciable differences in charge profile (IEX) among any of the formulations with the exception of formulations of 3C-3F, These formulations showed the smallest % change in main peak (data not shown) which further supports these formulations having moderately improved stability. The addition of methionine (10 mM) to the tested formulations had a negligible effect (0.2-0.4% difference) on the prevention of aggregation through the testing period.

TABLE 5
Formulations used for Example 2.

| | | | | | | Turbidity |
|---|---|---|---|---|---|---|
| | | | | % mAb (UP-SEC) | | (A350) |
| | | | Anti- | 10 days @ 5° | 10 days @ | 10 days @ |
| Form. | Stabilizer | Surfactant | Oxidant | C. | 50° C. | 50° C. |
| 1A | 1.4% sucrose | PS20 | — | 99.63 | 94.71 | 0.017 |
| 1B | 1.4% sucrose | PS20 | 10 mM Met | 99.62 | 94.90 | 0.017 |
| 1C | 1.4% sucrose | P188 | — | 99.63 | 94.78 | 0.018 |
| 1D | 1.4% sucrose | P188 | 10 mM Met | 99.62 | 95.05 | 0.017 |
| 1E | 1.4% sucrose | P338 | — | 99.62 | 95.03 | 0.019 |
| 1F | 1.4% sucrose | P338 | 10 mM Met | 99.62 | 95.19 | 0.016 |
| 1G | 1.4% sucrose | P407 | — | 99.57 | 94.79 | 0.022 |
| 1H | 1.4% sucrose | P407 | 10 mM Met | 99.56 | 95.09 | 0.019 |
| 1J | 1.4% sucrose | TPGS | — | 99.52 | 94.72 | 0.019 |
| 1K | 1.4% sucrose | TPGS | 10 mM Met | 99.62 | 95.02 | 0.017 |
| 1M | 1.4% sucrose | X100² | — | 99.55 | 94.67 | 0.022 |
| 1N | 1.4% sucrose | X100 | 10 mM Met | 99.54 | 95.09 | 0.021 |
| 2A | 0.8% mannitol | PS20 | — | 99.62 | 94.02 | 0.016 |
| 2B | 0.8% mannitol | PS20 | 10 mM Met | 99.62 | 94.36 | 0.015 |
| 2C | 0.8% mannitol | P188 | — | 99.62 | 94.34 | 0.016 |
| 2D | 0.8% mannitol | P188 | 10 mM Met | 99.62 | 94.66 | 0.015 |
| 2E | 0.8% mannitol | P338 | — | 99.63 | 94.44 | 0.017 |
| 2F | 0.8% mannitol | P338 | 10 mM Met | 99.61 | 94.75 | 0.016 |
| 2G | 0.8% mannitol | P407 | — | 99.54 | 94.34 | 0.020 |
| 2H | 0.8% mannitol | P407 | 10 mM Met | 99.56 | 94.65 | 0.020 |
| 2J | 0.8% mannitol | TPGS | — | 99.62 | 94.26 | 0.017 |
| 2K | 0.8% mannitol | TPGS | 10 mM Met | 99.63 | 94.53 | 0.016 |
| 2M | 0.8% mannitol | X100 | — | 99.55 | 94.19 | 0.020 |
| 2N | 0.8% mannitol | X100 | 10 mM Met | 99.55 | 94.55 | 0.019 |
| 3A | 1.4% trehalose | PS20 | — | 99.62 | 94.58 | 0.017 |
| 3B | 1.4% trehalose | PS20 | 10 mM Met | 99.63 | 94.87 | 0.015 |
| 3C | 1.4% trehalose | P188 | — | 99.63 | 94.80 | 0.016 |
| 3D | 1.4% trehalose | P188 | 10 mM Met | 99.62 | 95.09 | 0.015 |
| 3E | 1.4% trehalose | P338 | — | 99.63 | 94.88 | 0.016 |
| 3F | 1.4% trehalose | P338 | 10 mM Met | 99.62 | 95.22 | 0.016 |
| 3G | 1.4% trehalose | P407 | — | 99.55 | 94.72 | 0.033 |
| 3H | 1.4% trehalose | P407 | 10 mM Met | 99.56 | 95.08 | 0.025 |
| 3J | 1.4% trehalose | TPGS | — | 99.55 | 94.67 | 0.018 |
| 3K | 1.4% trehalose | TPGS | 10 mM Met | 99.62 | 94.89 | 0.016 |
| 3M | 1.4% trehalose | X100 | — | 99.61 | 94.72 | 0.022 |
| 3N | 1.4% trehalose | X100 | 10 mM Met | 99.52 | 95.01 | 0.019 |
| 4A | 1.4% HPβC | PS20 | — | 99.63 | 94.06 | 0.018 |
| 4B | 1.4% HPβC | PS20 | 10 mM Met | 99.62 | 94.62 | 0.018 |
| 4C | 1.4% HPβC | P188 | — | 99.62 | 94.06 | 0.018 |
| 4D | 1.4% HPβC | P188 | 10 mM Met | 99.63 | 94.49 | 0.016 |
| 4E | 1.4% HPβC | P338 | — | 99.62 | 94.20 | 0.018 |
| 4F | 1.4% HPβC | P338 | 10 mM Met | 99.62 | 94.55 | 0.018 |
| 4G | 1.4% HPβC | P407 | — | 99.53 | 94.14 | 0.026 |
| 4H | 1.4% HPβC | P407 | 10 mM Met | 99.56 | 94.54 | 0.022 |
| 4J | 1.4% HPβC | TPGS | — | 99.63 | 94.05 | 0.019 |
| 4K | 1.4% HPβC | TPGS | 10 mM Met | 99.51 | 94.56 | 0.016 |
| 4M | 1.4% HPβC | X100 | — | 99.55 | 94.12 | 0.025 |
| 4N | 1.4% HPβC | X100 | 10 mM Met | 99.52 | 94.49 | 0.022 |
| 5A | 0.8% proline | PS20 | — | 99.62 | 94.03 | 0.018 |
| 5B | 0.8% proline | PS20 | 10 mM Met | 99.51 | 94.42 | 0.016 |
| 5C | 0.8% proline | P188 | — | 99.64 | 94.49 | 0.018 |
| 5D | 0.8% proline | P188 | 10 mM Met | 99.63 | 94.65 | 0.016 |
| 5E | 0.8% proline | P338 | — | 99.63 | 94.38 | 0.018 |
| 5F | 0.8% proline | P338 | 10 mM Met | 99.62 | 94.75 | 0.017 |
| 5G | 0.8% proline | P407 | — | 99.55 | 94.32 | 0.022 |
| 5H | 0.8% proline | P407 | 10 mM Met | 99.54 | 94.68 | 0.020 |
| 5J | 0.8% proline | TPGS | — | 99.62 | 94.36 | 0.018 |
| 5K | 0.8% proline | TPGS | 10 mM Met | 99.53 | 94.53 | 0.016 |
| 5M | 0.8% proline | X100 | — | 99.53 | 94.45 | 0.022 |
| 5N | 0.8% proline | X100 | 10 mM Met | 99.54 | 94.63 | 0.020 |
| 6A | 0.4% glycine | PS20 | — | 99.63 | 94.22 | 0.016 |
| 6B | 0.4% glycine | PS20 | 10 mM Met | 99.53 | 94.60 | 0.015 |
| 6C | 0.4% glycine | P188 | — | 99.63 | 94.45 | 0.015 |
| 6D | 0.4% glycine | P188 | 10 mM Met | 99.51 | 94.71 | 0.014 |
| 6E | 0.4% glycine | P338 | — | 99.62 | 94.57 | 0.016 |
| 6F | 0.4% glycine | P338 | 10 mM Met | 99.63 | 94.70 | 0.016 |
| 6G | 0.4% glycine | P407 | — | 99.56 | 94.46 | 0.019 |
| 6H | 0.4% glycine | P407 | 10 mM Met | 99.56 | 94.72 | 0.020 |
| 6J | 0.4% glycine | TPGS | — | 99.62 | 94.33 | 0.016 |
| 6K | 0.4% glycine | TPGS | 10 mM Met | 99.61 | 94.76 | 0.016 |
| 6M | 0.4% glycine | X100 | — | 99.55 | 94.39 | 0.020 |
| 6N | 0.4% glycine | X100 | 10 mM Met | 99.55 | 94.61 | 0.021 |

¹All formulations comprised 5 mg/mL pembrolizumab, 10 mM Histidine buffer and 0.004% PS80, in addition to the excipients listed.

### Example 3

Evaluation of the Stability of Low Concentration Pembrolizumab Formulations with Alkyl Saccharide Stabilizing Surfactants in Combination with Methionine

Alkyl saccharides have been shown to be suitable stabilizing surfactants for protein formulations in the literature through maximizing colloidal stability; however, alkyl saccharides have been primarily studied with IgG1 proteins.

In order to investigate the impact of alkyl saccharide stabilizing surfactants on low concentration pembrolizumab (IgG4) formulations, several formulations containing n-dodecyl β-D-maltoside (DDM) and n-octyl β-D-maltoside (OM) were prepared and tested as described below. For formulations comprising DDM, a pembrolizumab (20 mg/mL)/DDM (0.16 mg/mL) stock solution in 10 mM histidine (pH 5.5) was prepared as described above in Example 2. Formulations 1-6 (P & Q) were prepared in a 96-well plate by spiking stock solutions (prepared in 10 mM histidine, pH 5.5 buffer) of the following excipients: sucrose (5% w/v), mannitol (5% w/v), trehalose (5% w/v), HPβC (5% w/v), proline (5% w/v), glycine (5% w/v), and methionine (2% w/v) into the pembrolizumab/DDM stock solution to achieve the target compositions (QS to 1 mL with histidine pH 5.5 buffer) listed in Table 6. Formulations 1-6 (R & S) were prepared in a 96-well plate by combining stock solutions of protein (2% w/v), OM (5% w/v), stabilizer (see Table 3), and methionine (2% w/v) followed by QS to 1 mL with histidine buffer (pH 5.5). All stock solutions used for formulations were filtered through Millapore Express® PLUS Stericup® 0.22 μm PES filters prior to use. The well plate was covered with a 96-well silicone sealing mat and then was vacuum sealed (2×) in moisture barrier bags to minimize potential evaporation. Samples were staged in 5° C. and 50° C. environmental stability chambers for time period of 10 days.

TABLE 6
Low Concentration Pembrolizumab Formulations with Non-ionic Surfactants

| | | | Anti- | | | | Anti- |
|---|---|---|---|---|---|---|---|
| Form.¹ | Stabilizer | Surfactant | Oxidant | Form.¹ | Stabilizer | Surfactant | Oxidant |
| 1P | 1 4% sucrose | DDM² | — | 4P | 1 4% HPβC | DDM | — |
| 1Q | 1.4% sucrose | DDM | 10 mM Met | 4Q | 1.4% HPβC | DDM | 10 mM Met |
| 1R | 1.4% sucrose | OM³ | — | 4R | 1.4% HPβC | OM | — |
| 1S | 1.4% sucrose | OM | 10 mM Met | 4S | 1.4% HPβC | OM | 10 mM Met |
| 2P | 0.8% mannitol | DDM | — | 5P | 0.8% proline | DDM | — |
| 2Q | 0.8% mannitol | DDM | 10 mM Met | 5Q | 0.8% proline | DDM | 10 mM Met |
| 2R | 0.8% mannitol | OM | — | 5R | 0.8% proline | OM | — |
| 2S | 0.8% mannitol | OM | 10 mM Met | 5S | 0.8% proline | OM | 10 mM Met |
| 3P | 1.4% trehalose | DDM | — | 6P | 0.4% glycine | DDM | — |
| 3Q | 1.4% trehalose | DDM | 10 mM Met | 6Q | 0.4% glycine | DDM | 10 mM Met |
| 3R | 1.4% trehalose | OM | — | 6R | 0.4% glycine | OM | — |
| 3S | 1.4% trehalose | OM | 10 mM Met | 6S | 0.4% glycine | OM | 10 mM Met |

¹All formulations comprised 5 mg/mL pembrolizumab and 10 mM histidine buffer (pH 5.5) in addition to the excipients listed.
²0.004% DDM.
³0.106% OM.

Each of the test formulations was visually inspected for any change in color or precipitate over the course of the 10 day testing period (data not shown). Additionally, stability of the formulations was evaluated using concentration (A280), turbidity (A350), dynamic light scattering (DLS), UP-SEC (to assess purity) and HP-IEX (charge profile) after the 10-day stability period. Similar to formulations in Table 2, all formulations showed an increase in % HMW species (UP-SEC, Table 7) and subsequent decrease in % monomer (data not shown) after the stability testing time period. Formulations comprising sucrose, trehalose and HPβD in combination with DDM showed the smallest change in % HMW species over the stability time period. On the contrary, formulations comprising mannitol in combination with DDM showed the poorest stability (largest growth in % HMW species) over 10 days @ 50° C. For a given stabilizer (i.e. sucrose, mannitol, trehalose, HPβD, proline, & glycine), formulations comprising DDM and OM showed very similar stability as reflected in the UP-SEC and turbidity data (Table 7). There were no appreciable differences in charge profile (IEX, data not shown) among any of the formulations. As shown in Table 7 from the SEC and turbidity data as well as DLS data (not shown), the addition of methionine (10 mM) to the tested formulations had a minimal effect (<0.6% difference, UP-SEC) on the prevention of aggregation through the testing period.

TABLE 7
Formulations used for Example 3.

| | | | | | | Turbidity |
|---|---|---|---|---|---|---|
| | | | | % HMW (UP-SEC) | | (A350) |
| | | | Anti- | 10 days @ 5° | 10 days @ | 10 days @ |
| Form. | Stabilizer | Surfactant | Oxidant | C. | 50° C. | 50° C. |
| 1P | 1.4% sucrose | DDM | — | 0.46 | 5.80 | 0.018 |
| 1Q | 1.4% sucrose | DDM | 10 mM Met | 0.36 | 5.40 | 0.017 |
| 1R | 1.4% sucrose | OM | — | 0.46 | 5.98 | 0.019 |
| 1S | 1.4% sucrose | OM | 10 mM Met | 0.39 | 5.40 | 0.018 |
| 2P | 0.8% mannitol | DDM | — | 0.38 | 6.42 | 0.017 |
| 2Q | 0.8% mannitol | DDM | 10 mM Met | 0.37 | 6.00 | 0.016 |
| 2R | 0.8% mannitol | OM | — | 0.39 | 6.48 | 0.019 |
| 2S | 0.8% mannitol | OM | 10 mM Met | 0.45 | 6.14 | 0.018 |
| 3P | 1.4% trehalose | DDM | — | 0.37 | 5.93 | 0.016 |
| 3Q | 1.4% trehalose | DDM | 10 mM Met | 0.37 | 5.54 | 0.015 |
| 3R | 1.4% trehalose | OM | — | 0.45 | 6.02 | 0.020 |
| 3S | 1.4% trehalose | OM | 10 mM Met | 0.44 | 5.66 | 0.018 |
| 4P | 1.4% HPβC | DDM | — | 0.38 | 5.75 | 0.019 |
| 4Q | 1.4% HPβC | DDM | 10 mM Met | 0.47 | 5.47 | 0.017 |
| 4R | 1.4% HPβC | OM | — | 0.45 | 5.67 | 0.024 |
| 4S | 1.4% HPβC | OM | 10 mM Met | 0.40 | 5.28 | 0.020 |
| 5P | 0.8% proline | DDM | — | 0.38 | 6.31 | 0.018 |
| 5Q | 0.8% proline | DDM | 10 mM Met | 0.38 | 6.04 | 0.016 |
| 5R | 0.8% proline | OM | — | 0.45 | 6.39 | 0.021 |
| 5S | 0.8% proline | OM | 10 mM Met | 0.39 | 6.02 | 0.019 |
| 6P | 0.4% glycine | DDM | — | 0.38 | 6.23 | 0.016 |
| 6Q | 0.4% glycine | DDM | 10 mM Met | 0.37 | 5.97 | 0.016 |
| 6R | 0.4% glycine | OM | — | 0.46 | 6.34 | 0.018 |
| 6S | 0.4% glycine | OM | 10 mM Met | 0.44 | 6.08 | 0.017 |

¹All formulations comprised 5 mg/mL pembrolizumab and 10 mM Histidine buffer, in addition to the excipients listed.

### Example 4

Evaluation of the Stability of Low Concentration Pembrolizumab Formulations with Ionic Stabilizing Surfactants in Combination with Methionine

In order to investigate the impact of ionic stabilizing surfactants on low concentration pembrolizumab formulations, several formulations containing sodium lauryl sulfate (SLS) and N,N-dimethyldodecylamine oxide (DDAO) were prepared and tested as described below. SLS is an anionic surfactant while DDAO is a zwitterionic surfactant. For formulations comprising DDAO, a pembrolizumab (20 mg/mL)/DDAO (0.16 mg/mL) stock solution in 10 mM histidine (pH 5.5) was prepared as described above in Example 3. Formulations 1-6 (V & W) were prepared in a 96-well plate by spiking stock solutions (prepared in 10 mM histidine, pH 5.5 buffer) of the following excipients: sucrose (5% w/v), mannitol (5% w/v), trehalose (5% w/v), HPβC (5% w/v), proline (5% w/v), glycine (5% w/v), and methionine (2% w/v) into the pembrolizumab/DDAO stock solution to achieve the target compositions (QS to 1 mL with histidine pH 5.5 buffer) listed in Table 8. Formulations 1-6 (T & U) were prepared in a 96-well plate by combining stock solutions of protein (2% w/v), SLS (0.5% w/v), stabilizer (see Table 5), and methionine (2% w/v) followed by QS to 1 mL with histidine buffer (pH 5.5). All stock solutions used for formulations were filtered through Millapore Express® PLUS Stericup® 0.22 μm PES filters prior to use. The well plate was covered with a 96-well silicone sealing mat and then was vacuum sealed (2×) in moisture barrier bags to minimize potential evaporation. Samples were staged in 5° C. and 50° C. environmental stability chambers for time period of 10 days.

TABLE 8
Low Concentration Pembrolizumab Formulations with Ionic Surfactants.

| | | | Anti- | | | | Anti- |
|---|---|---|---|---|---|---|---|
| Form.¹ | Stabilizer | Surfactant | Oxidant | Form.¹ | Stabilizer | Surfactant | Oxidant |
| 1T | 1.4% sucrose | SLS² | — | 4T | 1.4% HPβC | SLS² | — |
| 1U | 1.4% sucrose | SLS | 10 mM Met | 4U | 1.4% HPβC | SLS | 10 mM Met |
| 1V | 1.4% sucrose | DDAO³ | — | 4V | 1.4% HPβC | DDAO³ | — |
| 1W | 1.4% sucrose | DDAO | 10 mM Met | 4W | 1.4% HPβC | DDAO | 10 mM Met |
| 2T | 0.8% mannitol | SLS² | — | 5T | 0.8% proline | SLS² | — |
| 2U | 0.8% mannitol | SLS | 10 mM Met | 5U | 0.8% proline | SLS | 10 mM Met |
| 2V | 0.8% mannitol | DDAO³ | — | 5V | 0.8% proline | DDAO³ | — |
| 2W | 0.8% mannitol | DDAO | 10 mM Met | 5W | 0.8% proline | DDAO | 10 mM Met |
| 3T | 1.4% trehalose | SLS² | — | 6T | 0.4% glycine | SLS² | — |
| 3U | 1.4% trehalose | SLS | 10 mM Met | 6U | 0.4% glycine | SLS | 10 mM Met |
| 3V | 1.4% trehalose | DDAO³ | — | 6V | 0.4% glycine | DDAO³ | — |
| 3W | 1.4% trehalose | DDAO | 10 mM Met | 6W | 0.4% glycine | DDAO | 10 mM Met |

¹All formulations comprised 5 mg/mL pembrolizumab and 10 mM histidine buffer (pH 5.5) in addition to the excipients listed.
²0.23% SLS.
³0.004% DDAO.

Each of the test formulations was visually inspected for any change in color or precipitate over the course of the 10 day testing period (data not shown). Additionally, stability of the formulations was evaluated using concentration (A280), turbidity (A350), dynamic light scattering (DLS), UP-SEC (to assess purity) and HP-IEX (charge profile) after the 10-day stability period.

All formulations comprising SLS [1-6 (T & U)] were visibly turbid upon removal from the stability chamber. On the contrary, formulations comprising DDAO [1-6 (V & W)] did not show any visible signs of turbidity. Formulations 1-6 (T & U) were not tested further. For formulations 1-6 (V & W), UP-SEC results demonstrated that there were no changes for any of the formulations that were stored at 5° C. during the 10-day time period. At 50° C., more significant changes were observed via UP-SEC (a decrease in % mAb with subsequent increase in % HMW and % LMW species) and HP-IEX (a decrease in % main and a significant increase in % pre-main, data not shown) for each of the formulations over the time period tested. DLS data (not shown) further supported the observed decrease in % mAb (with subsequent increase in % HMW species) where a small increase in radius (nm) and % polydispersity (% Pd) were observed after the stability time period. Formulations 4V and 4W (HPβD+DDAO with and without methionine) proved to be the least stable through the stability testing period according to UP-SEC data (lowest % monomer, Table 9). There were no appreciable differences in charge profile among any of the formulations (1-6 (V & W)) at 50° C. for the 10-day testing period (data not shown). As shown in Table 9, the addition of methionine (10 mM) to the tested formulations had minimal effect on the prevention of aggregation (<0.4% difference) through the testing period (UP-SEC and Turbidity (A350) results).

TABLE 9
Formulations used for Example 4.

| | | | | | | Turbidity |
|---|---|---|---|---|---|---|
| | | | | % monomer (UP-SEC) | | (A350) |
| | | | | 10 days @ | 10 days @ | 10 days @ |
| Form. | Stabilizer | Surfactant | Anti-Oxidant | 5° C. | 50° C. | 50° C. |
| 1T | 1.4% sucrose | SLS² | — | DNT; visibly turbid, precipitate observed | | |
| 1U | 1.4% sucrose | SLS | 10 mM Met | | | |
| 1V | 1.4% sucrose | DDAO³ | — | 99.56 | 94.66 | 0.022 |
| 1W | 1.4% sucrose | DDAO | 10 mM Met | 99.54 | 94.95 | 0.019 |
| 2T | 0.8% mannitol | SLS² | — | DNT; visibly turbid, precipitate observed | | |
| 2U | 0.8% mannitol | SLS | 10 mM Met | | | |
| 2V | 0.8% mannitol | DDAO³ | — | 99.54 | 94.21 | 0.023 |
| 2W | 0.8% mannitol | DDAO | 10 mM Met | 99.54 | 94.59 | 0.018 |
| 3T | 1.4% trehalose | SLS² | — | DNT; visibly turbid, precipitate observed | | |
| 3U | 1.4% trehalose | SLS | 10 mM Met | | | |
| 3V | 1.4% trehalose | DDAO³ | — | 99.53 | 94.61 | 0.021 |
| 3W | 1.4% trehalose | DDAO | 10 mM Met | 99.54 | 94.97 | 0.020 |
| 4T | 1.4% HPβC | SLS² | — | DNT; visibly turbid, precipitate observed | | |
| 4U | 1.4% HPβC | SLS | 10 mM Met | | | |
| 4V | 1.4% HPβC | DDAO³ | — | 99.54 | 94.03 | 0.023 |
| 4W | 1.4% HPβC | DDAO | 10 mM Met | 99.55 | 94.38 | 0.020 |
| 5T | 0.8% proline | SLS² | — | DNT; visibly turbid, precipitate observed | | |
| 5U | 0.8% proline | SLS | 10 mM Met | | | |
| 5V | 0.8% proline | DDAO³ | — | 99.54 | 94.14 | 0.023 |
| 5W | 0.8% proline | DDAO | 10 mM Met | 99.54 | 94.53 | 0.019 |
| 6T | 0.4% glycine | SLS² | — | DNT; visibly turbid, precipitate observed | | |
| 6U | 0.4% glycine | SLS | 10 mM Met | | | |
| 6V | 0.4% glycine | DDAO³ | — | 99.55 | 94.28 | 0.020 |
| 6W | 0.4% glycine | DDAO | 10 mM Met | 99.55 | 94.54 | 0.020 |

1All formulations comprised 5 mg/mL pembrolizumab and 10 mM histidine buffer (pH 5.5) in addition to the excipients listed.
²0.23% SLS.
³0.004% DDAO.

### Example 5

Evaluation of the Viscosity of High Concentration Pembrolizumab Formulations with Amino Acid-Based Stabilizers

An exploratory formulation study was performed to evaluate the viscosity reducing ability of amino acids on high concentration solutions of pembrolizumab in the range of 200-250 mg/mL. The antibody solutions were prepared in 10 mM Histidine buffer (pH 5.5). The amino acid excipients were weighed and added into the respective antibody solution and stirred until dissolved to yield the final formulation. The final pH after the excipient addition was not adjusted. The formulation compositions are shown in Table 10 below.

TABLE 10
High Concentration Pembrolizumab Formulations with
Amino Acid Excipients.

| | Viscosity Reducing | | | |
|---|---|---|---|---|
| | Agent/Stabilizer | Pembrolizumab | | |
| Form. | and Conc. (w/v) | Conc. | | Buffer |
| 1 | N/A | 252 | mg/mL | 10 mM Histidine |
| 2 | 2.1% L-Arginine HCl | 252 | mg/mL | 10 mM Histidine |
| 3 | 3.1% L-Arginine HCl | 252 | mg/mL | 10 mM Histidine |
| 4 | N/A | 203-230 | mg/mL | 10 mM Histidine |
| 5 | 1.7% Proline | 203-230 | mg/mL | 10 mM Histidine |
| 6 | 2.3% L-Histidine | 203-230 | mg/mL | 10 mM Histidine |

Each of the test formulations in Table 10 were visually inspected for changes in coloration or precipitate formation (data not shown). Additionally, the viscosity of all the formulations were measured using MVROC or INITIUM and all measurements were made at 20° C. The viscosity of the formulations containing the amino acid excipient were compared against a control formulation formulated at the same concentration of pembrolizumab in 10 mM Histidine buffer in the absence of any additional amino acid excipient (i.e., formulations 2 and 3 are compared against formulation 1; and formulations 5 and 6 are compared against formulation 3). As shown in Table 11 below, the amino acids tested helped lower the viscosity of the high concentration pembrolizumab formulations. All the formulations containing the amino acid excipients helped lower the viscosity of the formulations to less than 100 cps.

TABLE 11
Viscosity Lowering Effect of Amino Acid Excipients of Pembrolizumab.

| | Viscosity Reducing | | | | |
|---|---|---|---|---|---|
| | Agent/Stabilizer and | Pembrolizumab | | Viscosity @ | |
| Form. | Conc. (w/v) | Conc. | | 20° C. | |
| 1 | N/A | 252 | mg/mL | ~150-200 | cps |
| 2 | 2.1% L-Arginine HCl | 252 | mg/mL | 80-100 | cps |
| 3 | 3.1% L-Arginine HCl | 252 | mg/mL | 80-90 | cps |
| 4 | N/A | 203-230 | mg/mL | 110-120 | cps |
| 5 | 1.7% Proline | 203-230 | mg/mL | 60-70 | cps |
| 6 | 2.3% L-Histidine | 203-230 | mg/mL | 60-70 | cps |

What is claimed is:

1. An anti-human PD-1 antibody formulation, comprising:
a) about 5 mg/mL to about 250 mg/mL of an anti-human PD-1 antibody, or antigen binding fragment thereof;
b) about 5 mM to about 20 mM buffer;
c) about 1.5 to about 8.0% weight/volume (w/v) stabilizer selected from the group consisting of: a non-reducing sugar, (2-hydroxypropyl)-β-cyclodextrin, mannitol, sorbitol, L-arginine, a pharmaceutically acceptable salt of L-arginine, L-proline, a pharmaceutically acceptable salt of L-proline, L-histidine, a pharmaceutically acceptable salt of L-histidine, glycine, and a pharmaceutically acceptable salt of glycine;
d) a surfactant selected from:
i) about 0.005% w/v to about 0.60% w/v non-ionic surfactant,
ii) about 0.23% w/v to about 1.15% w/v ionic surfactant, or
iii) about 0.005% w/v to about 0.20% w/v dimethyl-dodecylamine oxide (DDAO); and
e) about 1 mM to about 30 mM anti-oxidant.

2. (canceled)

3. The anti-human PD-1 antibody formulation of claim 1, wherein:
a) the stabilizer is selected from the group consisting of:
i) about 6% to about 8% weight/volume (w/v) sucrose, trehalose or (2-hydroxypropyl)-β-cyclodextrin;
ii) about 3% to about 5% w/v mannitol, sorbitol, L-arginine, a pharmaceutically acceptable salt of L-arginine, L-proline, or a pharmaceutically acceptable salt of L-proline;
iii) about 1.8% to about 2.2% w/v glycine, or a pharmaceutically acceptable salt thereof;
iv) about 1.5% to about 1.9% w/v L-proline, or a pharmaceutically acceptable sale of L-proline;
v) about 1.9% to about 3.3% w/v L-arginine, or a pharmaceutically acceptable salt of L-arginine; and
vi) about 2% to about 3% L-histidine, or a pharmaceutically acceptable salt of L-histidine; and
b) the antioxidant is about 1 mM to about 20 mM L-methionine or a pharmaceutically acceptable salt thereof.

4. The anti-human PD-1 antibody formulation of claim 1, wherein the stabilizer is about 1.5% to 1.9% w/v L-proline, or a pharmaceutically acceptable sale of L-proline.

5. (canceled)

6. The anti-human PD-1 antibody formulation of claim 1, comprising greater than 200 mg/mL of the anti-human PD-1 antibody, or antigen binding fragment thereof.

7. (canceled)

8. The anti-human PD-1 antibody formulation of claim 1, wherein the surfactant is:
a) about 0.23% w/v to about 1.15% w/v sodium dodecyl sulfate;
b) about 0.005% w/v to about 0.60% w/v non-ionic surfactant, which is selected from the group consisting of: poloxamer 338 (P338), poloxamer 407 (P407), vitamin E D-α-tocopherol polyethylene glycol succinate (TPGS), n-dodecyl β-D-maltoside (DDM) and n-octyl β-D-maltoside (OM), or
c) about 0.005% w/v to about 0.20% w/v dimethyl-dodecylamine oxide (DDAO).

9. The anti-human PD-1 antibody formulation of claim 1, wherein the surfactant is about 0.01% to about 0.03% w/v poloxamer 338 (P338).

10. The anti-human PD-1 antibody formulation of claim 1, wherein the surfactant is about 0.01% to about 0.03% w/v poloxamer 407 (P407).

11. The anti-human PD-1 antibody formulation of claim 1, wherein the surfactant is about 0.01% to about 0.03% w/v vitamin E D-α-tocopherol polyethylene glycol succinate (TPGS).

12. The anti-human PD-1 antibody formulation of claim 1, wherein the surfactant is about 0.01% to about 0.03% w/v n-dodecyl β-D-maltoside (DDM).

13. The anti-human PD-1 antibody formulation of claim 1, wherein the surfactant is about 0.4% to about 0.6% w/v n-octyl β-D-maltoside (OM).

14. The anti-human PD-1 antibody formulation of claim 1, wherein the surfactant is about 0.01% to about 0.03% w/v dimethyl-dodecylamine oxide (DDAO).

15. (canceled)

16. (canceled)

17. The anti-human PD-1 antibody formulation of claim 1, wherein the formulation comprises:
a) about 200 mg/mL to about 250 mg/mL of an anti-human PD-1 antibody, or antigen binding fragment thereof;
b) about 5 mM to about 20 mM histidine buffer;
c) a stabilizer selected from the group consisting of:
i) about 6% to about 8% weight/volume (w/v) sucrose, trehalose or (2-hydroxypropyl)-β-cyclodextrin;
ii) about 3% to about 5% w/v mannitol, sorbitol, L-arginine, a pharmaceutically acceptable salt of L-arginine, L-proline, or a pharmaceutically acceptable salt of L-proline;
iii) about 1.8 to about 2.2% w/v glycine, or a pharmaceutically acceptable salt thereof;
iv) about 1.5% to about 1.9% w/v L-proline, or a pharmaceutically acceptable sale of L-proline;
v) about 1.9% to about 3.3% w/v L-arginine, or a pharmaceutically acceptable salt of L-arginine; and
vi) about 2% to about 3% L-histidine, or a pharmaceutically acceptable salt of L-histidine;
d) about 0.01% w/v to about 0.10% w/v polysorbate 80; and
e) about 1 mM to about 20 mM L-methionine, or a pharmaceutically acceptable salt thereof.

18. The anti-human PD-1 antibody formulation of claim 17, wherein the formulation further comprises from about 1% to about 3% w/v of a viscosity reducing agent selected from the group consisting of:
a) L-arginine, or a pharmaceutically acceptable thereof,
b) L-lysine, or a pharmaceutically acceptable thereof,
c) L-histidine, or a pharmaceutically acceptable thereof, and
d) L-glutamine, or a pharmaceutically acceptable thereof.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. The anti-human PD-1 antibody formulation of claim 1, wherein the formulation further comprises a metal chelator.

36. (canceled)

37. (canceled)

38. (canceled)

39. (canceled)

40. The anti-human PD-1 antibody formulation of claim 1, wherein the anti-human PD-1 antibody or antigen binding fragment thereof comprises three light chain CDRs comprising CDRL1 of SEQ ID NO:1, CDRL2 SEQ ID NO:2 and CDRL3 of SEQ ID NO:3 and three heavy chain CDRs of CDRH1 of SEQ ID NO:6, CDRH2 of SEQ ID NO:7 and CDRH3 SEQ ID NO:8.

41. The anti-human PD-1 antibody formulation of claim 1, wherein the anti-human PD-1 antibody or antigen binding fragment thereof comprises a VL region which comprises the amino acid sequence set forth in SEQ ID NO:4, and a VH region which comprises the amino acid sequence set forth in SEQ ID NO:9.

42. The anti-human PD-1 antibody formulation of claim 1, wherein the formulation comprises a light chain comprising or consisting of a sequence of amino acids as set forth in SEQ ID NO:5 and a heavy chain comprising or consisting of a sequence of amino acids as set forth in SEQ ID NO:10.

43. The anti-human PD-1 antibody formulation of claim 1, wherein the formulation comprises an anti-human PD-1 antibody that is pembrolizumab.

44. A method of treating cancer in a human patient in need thereof, the method comprising administering an effective amount of the anti-human PD-1 antibody formulation of claim 1.

45. (canceled)

46. (canceled)

47. (canceled)

48. (canceled)

49. (canceled)

50. (canceled)

51. (canceled)

52. The method of claim 44, wherein the anti-human PD-1 antibody formulation is administered by subcutaneous administration.

53. (canceled)

54. (canceled)

55. (canceled)
